(19) United States
(12) Patent Application Publication
(10) Pub. No.: US 2012/0283111 A1
(43) Pub. Date: Nov. 8, 2012

(54) Bacterial mRNA screen strategy for novel pesticide-encoding nucleic acid molecule discovery

(75) Inventors: Andre R. Abad, Johnston, IA (US); Deirdre M. Kapka-Kitzman, Ankeny, IA (US)

(73) Assignee: PIONEER HI-BRED INTERNATIONAL, INC., Johnston, IA (US)

(21) Appl. No.: 13/459,280

(22) Filed: Apr. 30, 2012

Related U.S. Application Data
(60) Provisional application No. 61/481,442, filed on May 2, 2011.

(51) Int. Cl. C40B 30/00; C12Q 1/68

(52) U.S. Cl. 506/7; 435/6.11

(57) ABSTRACT

Methods are provided for isolating and identifying novel pesticide-encoding nucleic acid molecules from bacterial strains identified as having a pesticidal polypeptide. The methods involve selecting a bacterial strain having or suspected of having at least one pesticide-encoding plasmid, curing the at least one pesticide-encoding plasmid from the bacterial strain and performing a subtractive hybridization to obtain plasmid mRNA encoding the pesticidal polypeptide. The plasmid mRNA can be used to make a cDNA library from which the pesticidal polypeptide can be isolated and subsequently identified.

### CROSS REFERENCE TO RELATED APPLICATIONS

This application claims priority to U.S. Provisional Application No. 61/481,442, filed May 2, 2011, which is hereby incorporated herein in its entirety by reference.

### FIELD OF THE INVENTION

The present invention generally relates to methods for isolating and identifying novel pesticide-encoding nucleic acid molecules, and more particularly relates to methods for isolating and identifying pesticide-encoding nucleic acid molecules from prokaryotes such as bacteria by subtractive hybridization.

### BACKGROUND OF THE INVENTION

Pests, such as insect pests, are a major factor in the loss of the world's agricultural crops. For example, corn rootworm feeding damage and boll weevil damage can be economically devastating to agricultural producers. Insect pest-related agricultural crop loss from corn rootworm alone has reached one billion dollars a year.

Traditionally, the primary methods for controlling insect pests, such as corn rootworm, are crop rotation and application of broad-spectrum, synthetic, chemical pesticides. However, consumers and government regulators alike are becoming increasingly concerned with environmental hazards associated with producing and using chemical pesticides. Because of such concerns, regulators have banned or limited the use of some of the more hazardous chemical pesticides. Thus, there is substantial interest in developing alternatives to chemical pesticides that present a lower risk of pollution and environmental hazards and that provide greater target specificity than is characteristic of chemical pesticides.

Certain species in the genus Bacillus have polypeptides that possess pesticidal activity against a broad range of insect pests including those in the orders Lepidoptera, Diptera, Coleoptera, Hemiptera and others. For example, Bacillus thuringiensis and Bacillus popilliae are among the most successful species having pesticidal activity discovered to date. Such pesticidal activity also has been attributed to strains of Bacillus larvae, Bacillus lentimorbus, Bacillus sphaericus and Bacillus cereus. See, Biotechnology Handbook 2: Bacillus (Harwood ed., Plenum Press 1989); and Int'l Patent Application Publication No. WO 96/10083.

Pesticidal polypeptides from Bacillus spp. include the crystal (Cry) endotoxins, cytolytic (Cyt) endotoxins, vegetative proteins (VIPs) and the like. See, e.g., Bravo et al. (2007) Toxicon 49:423-435. The Cry endotoxins (also called δ-endotoxins) are pesticidal polypeptides that have been isolated from strains of B. thuringiensis. The Cry endotoxins initially are produced in an inactive protoxin form, which are proteolytically converted into an active endotoxin through the action of proteases in an insect's gut. Once active, the endotoxin binds to the gut epithelium and forms cation-selective channels that cause cell lysis and subsequent death. See, Carroll et al. (1997) J. Invertebr. Pathol. 70:41-49; Oppert (1999) Arch. Insect Biochem. Phys. 42:1-12; and Rukmini et al. (2000) Biochimie 82:109-116. A common characteristic of the Cry endotoxins is their expression during the stationary phase of growth, as they generally accumulate in a mother cell compartment to form a crystal inclusion that can account for 23-30% of the dry weight of sporulated cells.

Recently, scientists have developed crop plants with enhanced insect resistance by genetically engineering the crop plants with pesticide-encoding nucleic acid molecules such as the Cry endotoxins. For example, corn and cotton plants have been genetically engineered to produce Cry endotoxins. See, e.g., Aronson (2002) Cell Mol. Life Sci. 59:417-425; and Schnepf et al. (1998) Microbiol. Mol. Biol. Rev. 62:775-806. Other crops, including potatoes, have been genetically engineered to contain Cry endotoxins. See, e.g., Hussein et al. (2006) J. Chem. Ecol. 32:1-8; and Kalushkov & Nedved (2005) J. Appl. Entomol. 129:401-406. These plants are now widely used in American agriculture and provide farmers with an environmentally friendly alternative to chemical pesticides. Because of these successes, researchers continue to search for novel pesticide-encoding nucleic acid molecules such as additional cry genes. Therefore, new methods for efficiently identifying novel pesticide-encoding nucleic acid molecules are needed in the art.

### BRIEF SUMMARY OF THE INVENTION

Methods are provided for isolating and identifying novel pesticide-encoding nucleic acid molecules. The methods provide for enriching for pesticide-encoding nucleic acid molecules, particularly plasmid mRNA, in a bacterial strain identified as having a pesticidal polypeptide. The methods involve selecting a bacterial strain having or suspected of having at least one pesticidal protein, curing the plasmid from the bacterial strain and performing an in vitro or in silico subtractive hybridization to obtain plasmid mRNA. In one embodiment, the substractive hybridization can be performed in vitro or in silico. For in vitro methods, the plasmid mRNA can be used to make a cDNA library from which the pesticidal polypeptide can be isolated and subsequently identified. For in silico methods, the pesticidal coding sequence can be identified.

Compositions are provided, which include isolated pesticide-encoding nucleic acid molecules, variants and fragments thereof, as well as pesticidal polypeptides, variants and fragments thereof. Organisms comprising the pesticide-encoding nucleic acid molecules are also provided.

The methods and compositions of the invention therefore find use in discovering nucleic acid molecules that encode pesticidal polypeptides and the pesticidal polypeptides for protecting plants from pests, especially insect pests.

### BRIEF DESCRIPTION OF THE DRAWINGS

FIG. 1 presents temporal RNA expression for Bacillus thuringiensis strain DP1019 in samples collected every 4 hours between 8 and 32 hours post-inoculation.

### DETAILED DESCRIPTION OF THE INVENTION

Overview

Methods are provided for isolating and identifying novel nucleic acid molecules encoding pesticidal polypeptides. The methods involve identifying a bacteria containing, or suspected of containing, a pesticidal protein. Genes encoding the endotoxins are located mainly on large plasmids, although chromosomally encoded endotoxins have been reported. See, Ben-Dov et al. (1996) Appl. Environ. Microbiol. 62:3140-3145; Berry et al. (2002) Appl. Environ. Microbiol. 68:5082-5095; Gonzáles et al. (1981) Plasmid 5:351-365; Lereclus et al. (1982) Mol. Gen. Genet. 186:391-398; and Trisrisook et al. (1990) Appl. Environ. Microbiol.

56:1710-1716. Many cry gene-containing plasmids appear to be conjugative in nature. Accordingly, the plasmid or plasmids are cured from a population of the bacteria leaving only chromosomal DNA. This cured population subsequently can be used for assessing differential gene expression in an uncured (i.e., wild-type) population of the same bacterial strain. As such, nucleic acid molecules (e.g., total mRNA) can be isolated from the cured population (negative control) as well as from the corresponding uncured population (target). In vitro or in silico subtractive hybridization can be used to identify plasmid-encoding genes. These plasmid-encoded genes can be screened to identify the coding sequence for the pesticidal protein.

As used herein, “pesticidal protein” means a polypeptide that is capable of inhibiting growth, feeding, reproduction, or is capable of killing of the pest. One of skill in the art understands that not all substances or mixtures thereof are equally effective against all pests. Of particular interest herein are pesticidal polypeptides that act as insecticides and thus have biological activity against insect pests.

As used herein, “pest” means an organism that interferes with or is harmful to plant development and/or growth. Examples of pests include, but are not limited to, algae, arachnids (e.g., acarids including mites and ticks), bacteria (e.g., plant pathogens including Xanthomonas spp. and Pseudomonas spp.), crustaceans (e.g., pillbugs and sowbugs); fungi (e.g., members in the phylum Ascomycetes or Basidiomycetes, and fungal-like organisms including Oomycetes such as Pythium spp. and Phytophthora spp.), insects, mollusks (e.g., snails and slugs), nematodes (e.g., soil-transmitted nematodes including Clonorchis spp., Fasciola spp., Heterodera spp., Globodera spp., Opisthorchis spp. and Paragonimus spp.), protozoans (e.g., Phytomonas spp.), viruses (e.g., Comovirus spp., Cucumovirus spp., Cytorhabdovirus spp., Luteovirus spp., Nepovirus spp., Potyvirus spp., Tobamovirus spp., Tombusvirus spp. and Tospovirus spp.) and weeds.

Of particular interest herein are insect pests. As used herein, “insect pest” means an organism in the phylum Arthropoda that interferes with or is harmful to plant development and/or growth, and more specifically means an organism in the class Insecta. The class Insecta can be divided into two groups historically treated as subclasses: (1) wingless insects, known as Apterygota; and (2) winged insects, known as Pterygota. Examples of insect pests include, but are not limited to, insects in the orders Coleoptera, Diptera, Hemiptera, Homoptera, Hymenoptera, Isoptera, Lepidoptera, Mallophaga, Orthroptera, Thysanoptera, Dermaptera, Isoptera, Anoplura, Siphonaptera, Trichoptera and Thysanura. While technically not insects, arthropods such as arachnids, especially in the order Acari, are included in “insect pest.”

The insect pests can be adults, larvae or even ova. A preferred developmental stage for testing for pesticidal activity is larvae or other immature form of the insect pest. Methods of rearing insect larvae and performing bioassays are well known in the art. See, e.g., Czapla & Lang (1990) J. Econ. Entomol. 83:2480-2485; Griffith & Smith (1977) J. Aust. Ent. Soc. 16:366; Keiper & Foote (1996) Hydrobiologia 339:137-139; and U.S. Pat. No. 5,351,643. For example, insect pests can be reared in total darkness at about 20° C. to about 30° C. and from about 30% to about 70% relative humidity.

Insect pests include insects selected from the orders Coleoptera, Diptera, Hymenoptera, Lepidoptera, Mallophaga, Homoptera, Hemiptera, Orthoptera, Thysanoptera, Dermaptera, Isoptera, Anoplura, Siphonaptera, Trichoptera, etc., particularly Coleoptera and Lepidoptera.

Insects of the order Lepidoptera include, but are not limited to, armyworms, cutworms, loopers, and heliothines in the family Noctuidae Agrotis ipsilon Hufnagel (black cutworm); A. orthogonia Morrison (western cutworm); A. segetum Denis & Schiffermüller (turnip moth); A. subterranea Fabricius (granulate cutworm); Alabama argillacea Hübner (cotton leaf worm); Anticarsia gemmatalis Hübner (velvetbean caterpillar); Athetis mindara Barnes and McDunnough (rough skinned cutworm); Earias insulana Boisduval (spiny bollworm); E. vittella Fabricius (spotted bollworm); Egira (Xylomyges) curialis Grote (citrus cutworm); Euxoa messoria Harris (darksided cutworm); Helicoverpa armigera Hübner (American bollworm); H. zea Boddie (corn earworm or cotton bollworm); Heliothis virescens Fabricius (tobacco budworm); Hypena scabs Fabricius (green cloverworm); Hyponeuma taltula Schaus; (Mamestra configurata Walker (bertha armyworm); M. brassicae Linnaeus (cabbage moth); Melanchra picta Harris (zebra caterpillar); Mocis latipes Guenée (small mocis moth); Pseudaletia unipuncta Haworth (armyworm); Pseudoplusia includens Walker (soybean looper); Richia albicosta Smith (Western bean cutworm);Spodoptera frugiperda J E Smith (fall armyworm); S. exigua Hübner (beet armyworm); S. litura Fabricius (tobacco cutworm, cluster caterpillar); Trichoplusia ni Hübner (cabbage looper); borers, casebearers, webworms, coneworms, and skeletonizers from the families Pyralidae and Crambidae such as Achroia grisella Fabricius (lesser wax moth); Amyelois transitella Walker (naval orangeworm); Anagasta kuehniella Zeller (Mediterranean flour moth); Cadra cautella Walker (almond moth); Chilo partellus Swinhoe (spotted stalk borer); C. suppressalis Walker (striped stem/rice borer); C. terrenellus Pagenstecher (sugarcane stemp borer); Corcyra cephalonica Stainton (rice moth); Crambus caliginosellus Clemens (corn root webworm); C. teterrellus Zincken (bluegrass webworm); Cnaphalocrocis medinalis Guenée (rice leaf roller); Desmia funeralis Hübner (grape leaffolder); Diaphania hyalinata Linnaeus (melon worm); D. nitidalis Stoll (pickleworm); Diatraea flavipennella Box; D. grandiosella Dyar (southwestern corn borer), D. saccharalis Fabricius (surgarcane borer); Elasmopalpus lignosellus Zeller (lesser cornstalk borer); Eoreuma loftini Dyar (Mexican rice borer); Ephestia elutella Hübner (tobacco (cacao) moth); Galleria mellonella Linnaeus (greater wax moth); Hedylepta accepta Butler (sugarcane leafroller); Herpetogramma licarsisalis Walker (sod webworm); Homoeosoma electellum Hulst (sunflower moth); Loxostege sticticalis Linnaeus (beet webworm); Maruca testulalis Geyer (bean pod borer); Orthaga thyrisalis Walker (tea tree web moth); Ostrinia nubilalis Hübner (European corn borer); Plodia interpunctella Hübner (Indian meal moth); Scirpophaga incertulas Walker (yellow stem borer); Udea rubigalis Guenée (celery leaftier); and leafrollers, budworms, seed worms, and fruit worms in the family Tortricidae Acleris gloverana Walsingham (Western blackheaded budworm); A. variana Fernald (Eastern blackheaded budworm); Adoxophyes orana Fischer von Rösslerstamm (summer fruit tortrix moth); Archips spp. including A. argyrospila Walker (fruit tree leaf roller) and A. rosana Linnaeus (European leaf roller); Argyrotaenia spp.; Bonagota salubricola Meyrick (Brazilian apple leafroller); Choristoneura spp.; Cochylis hospes Walsingham (banded sunflower moth); Cydia latiferreana Walsingham (filbertworm); C. pomonella Linnaeus (codling moth); Endopiza viteana Clemens (grape berry moth); Eupoecilia ambiguella Hübner (vine moth); Grapholita molesta Busck (oriental fruit moth); Lobesia botrana Denis & Schiffermüller (European grape vine moth); Platynota flavedana Clemens (variegated leafroller); P. stultana Walsingham (omnivorous leafroller); Spilonota ocellana Denis & Schiffermüller (eyespotted bud moth); and Suleima helianthana Riley (sunflower bud moth).

Selected other agronomic pests in the order Lepidoptera include, but are not limited to, Alsophila pometaria Harris (fall cankerworm); Anarsia lineatella Zeller (peach twig borer); Anisota senatoria J. E. Smith (orange striped oakworm); Antheraea pernyi Guérin-Méneville (Chinese Oak Silkmoth); Bombyx mori Linnaeus (Silkworm); Bucculatrix thurberiella Busck (cotton leaf perforator); Colias eurytheme Boisduval (alfalfa caterpillar); Datana integerrima Grote & Robinson (walnut caterpillar); Dendrolimus sibiricus Tschetwerikov (Siberian silk moth), Ennomos subsignaria Hübner (elm spanworm); Erannis tiliaria Harris (linden looper); Erechthias flavistriata Walsingham (sugarcane bud moth); Euproctis chrysorrhoea Linnaeus (browntail moth); Harrisina americana Guérin-Méneville (grapeleaf skeletonizer); Heliothis subflexa Guenée; Hemileuca oliviae Cockrell (range caterpillar); Hyphantria cunea Drury (fall webworm); Keiferia lycopersicella Walsingham (tomato pinworm); Lambdina fiscellaria fiscellaria Hulst (Eastern hemlock looper); L. fiscellaria lugubrosa Hulst (Western hemlock looper); Leucoma salicis Linnaeus (satin moth); Lymantria dispar Linnaeus (gypsy moth); Malacosoma spp.; Manduca quinquemaculata Haworth (five spotted hawk moth, tomato hornworm); M. sexta Haworth (tomato hornworm, tobacco hornworm); Operophtera brumata Linnaeus (winter moth); Orgyia spp.; Paleacrita vernata Peck (spring cankerworm); Papilio cresphontes Cramer (giant swallowtail, orange dog); Phryganidia californica Packard (California oakworm); Phyllocnistis citrella Stainton (citrus leafminer); Phyllonorycter blancardella Fabricius (spotted tentiform leafminer); Pieris brassicae Linnaeus (large white butterfly); P. rapae Linnaeus (small white butterfly); P. napi Linnaeus (green veined white butterfly); Platyptilia carduidactyla Riley (artichoke plume moth); Plutella xylostella Linnaeus (diamondback moth); Pectinophora gossypiella Saunders (pink bollworm); Pontia protodice Boisduval & Leconte (Southern cabbageworm); Sabulodes aegrotata Guenée (omnivorous looper); Schizura concinna J. E. Smith (red humped caterpillar); Sitotroga cerealella Olivier (Angoumois grain moth); Telchin licus Drury (giant sugarcane borer); Thaumetopoea pityocampa Schiffermüller (pine processionary caterpillar); Tineola bisselliella Hummel (webbing clothesmoth); Tuta absoluta Meyrick (tomato leafminer) and Yponomeuta padella Linnaeus (ermine moth).

Of interest are larvae and adults of the order Coleoptera including weevils from the families Anthribidae, Bruchidae, and Curculionidae including, but not limited to: Anthonomus grandis Boheman (boll weevil); Cylindrocopturus adspersus LeConte (sunflower stem weevil); Diaprepes abbreviatus Linnaeus (Diaprepes root weevil); Hypera punctata Fabricius (clover leaf weevil); Lissorhoptrus oryzophilus Kuschel (rice water weevil); Metamasius hemipterus hemipterus Linnaeus (West Indian cane weevil); M. hemipterus sericeus Olivier (silky cane weevil); Sitophilus granarius Linnaeus (granary weevil); S. oryzae Linnaeus (rice weevil); Smicronyx fulvus LeConte (red sunflower seed weevil); S. sordidus LeConte (gray sunflower seed weevil); Sphenophorus maidis Chittenden (maize billbug);S. livis Vaurie (sugarcane weevil); Rhabdoscelus obscurus Boisduval (New Guinea sugarcane weevil); flea beetles, cucumber beetles, rootworms, leaf beetles, potato beetles, and leafminers in the family Chrysomelidae including, but not limited to: Chaetocnema ectypa Horn (desert corn flea beetle); C. pulicaria Melsheimer (corn flea beetle); Colaspis brunnea Fabricius (grape colaspis); Diabrotica barberi Smith & Lawrence (northern corn rootworm); D. undecimpunctata howardi Barber (southern corn rootworm); D. virgifera virgifera LeConte (western corn rootworm); Leptinotarsa decemlineata Say (Colorado potato beetle); Oulema melanopus Linnaeus (cereal leaf beetle); Phyllotreta cruciferae Goeze (corn flea beetle); Zygogramma exclamationis Fabricius (sunflower beetle); beetles from the family Coccinellidae including, but not limited to: Epilachna varivestis Mulsant (Mexican bean beetle); chafers and other beetles from the family Scarabaeidae including, but not limited to: Antitrogus parvulus Britton (Childers cane grub); Cyclocephala borealis Arrow (northern masked chafer, white grub); C. immaculata Olivier (southern masked chafer, white grub); Dermolepida albohirtum Waterhouse (Greyback cane beetle); Euetheola humilis rugiceps LeConte (sugarcane beetle); Lepidiota frenchi Blackburn (French's cane grub); Tomarus gibbosus De Geer (carrot beetle); T. subtropicus Blatchley (sugarcane grub); Phyllophaga crinita Burmeister (white grub); P. latifrons LeConte (June beetle); Popillia japonica Newman (Japanese beetle); Rhizotrogus majalis Razoumowsky (European chafer); carpet beetles from the family Dermestidae; wireworms from the family Elateridae, Eleodes spp., Melanotus spp. including M. communis Gyllenhal (wireworm); Conoderus spp.; Limonius spp.; Agriotes spp.; Ctenicera spp.; Aeolus spp.; bark beetles from the family Scolytidae; beetles from the family Tenebrionidae; beetles from the family Cerambycidae such as, but not limited to, Migdolus fryanus Westwood (longhorn beetle); and beetles from the Buprestidae family including, but not limited to, Aphanisticus cochinchinae seminulum Obenberger (leaf-mining buprestid beetle).

Adults and immatures of the order Diptera are of interest, including leafminers Agromyza parvicornis Loew (corn blotch leafminer); midges including, but not limited to: Contarinia sorghicola Coquillett (sorghum midge); Mayetiola destructor Say (Hessian fly); Neolasioptera murtfeldtiana Felt, (sunflower seed midge); Sitodiplosis mosellana Géhin (wheat midge); fruit flies (Tephritidae), Oscinella frit Linnaeus (frit flies); maggots including, but not limited to: Delia spp. including Delia platura Meigen (seedcorn maggot); D. coarctate Fallen (wheat bulb fly); Fannia canicularis Linnaeus, F. femoralis Stein (lesser house flies); Meromyza americana Fitch (wheat stem maggot); Musca domestica Linnaeus (house flies); Stomoxys calcitrans Linnaeus (stable flies)); face flies, horn flies, blow flies, Chrysomya spp.; Phormia spp.; and other muscoid fly pests, horse flies Tabanus spp.; bot flies Gastrophilus spp.; Oestrus spp.; cattle grubs Hypoderma spp.; deer flies Chrysops spp.; Melophagus ovinus Linnaeus (keds); and other Brachycera, mosquitoes Aedes spp.; Anopheles spp.; Culex spp.; black flies Prosimulium spp.; Simulium spp.; biting midges, sand flies, sciarids, and other Nematocera.

Included as insects of interest are those of the order Hemiptera such as, but not limited to, the following families: Adelgidae, Aleyrodidae, Aphididae, Asterolecaniidae, Cercopidae, Cicadellidae, Cicadidae, Cixiidae, Coccidae, Coreidae, Dactylopiidae, Delphacidae, Diaspididae, Eriococcidae, Flatidae, Fulgoridae, Issidae, Lygaeidae, Margarodidae, Membracidae, Miridae, Ortheziidae, Pentatomidae, Phoenicococcidae, Phylloxeridae, Pseudococcidae, Psyllidae, Pyrrhocoridae and Tingidae.

Agronomically important members from the order Hemiptera include, but are not limited to: Acrosternum hilare Say (green stink bug); Acyrthisiphon pisum Harris (pea aphid); Adelges spp. (adelgids); Adelphocoris rapidus Say (rapid plant bug); Anasa tristis De Geer (squash bug); Aphis craccivora Koch (cowpea aphid); A. fabae Scopoli (black bean aphid); A. gossypii Glover (cotton aphid, melon aphid); A. maidiradicis Forbes (corn root aphid); A. pomi De Geer (apple aphid); A. spiraecola Patch (spirea aphid); Aulacaspis tegalensis Zehntner (sugarcane scale); Aulacorthum solani Kaltenbach (foxglove aphid); Bemisia tabaci Gennadius (tobacco whitefly, sweetpotato whitefly); B. argentifolii Bellows & Perring (silverleaf whitefly); Blissus leucopterus leucopterus Say (chinch bug); Blostomatidae spp.; Brevicoryne brassicae Linnaeus (cabbage aphid); Cacopsylla pyricola Foerster (pear psylla); Calocoris norvegicus Gmelin (potato capsid bug); Chaetosiphon fragaefolii Cockerell (strawberry aphid); Cimicidae spp.; Coreidae spp.; Corythuca gossypii Fabricius (cotton lace bug); Cyrtopeltis modesta Distant (tomato bug); C. notatus Distant (suckfly); Deois flavopicta Stål (spittlebug); Dialeurodes citri Ashmead (citrus whitefly); Diaphnocoris chlorionis Say (honeylocust plant bug); Diuraphis noxia Kurdjumov/Mordvilko (Russian wheat aphid); Duplachionaspis divergens Green (armored scale); Dysaphis plantaginea Paaserini (rosy apple aphid); Dysdercus suturellus Herrich-Schäffer (cotton stainer); Dysmicoccus boninsis Kuwana (gray sugarcane mealybug); Empoasca fabae Harris (potato leafhopper); Eriosoma lanigerum Hausmann (woolly apple aphid); Erythroneoura spp. (grape leafhoppers); Eumetopina flavipes Muir (Island sugarcane planthopper); Eurygaster spp.; Euschistus servus Say (brown stink bug); E. variolarius Palisot de Beauvois (one-spotted stink bug); Graptostethus spp. (complex of seed bugs); and Hyalopterus pruni Geoffroy (mealy plum aphid); Icerya purchasi Maskell (cottony cushion scale); Labopidicola allii Knight (onion plant bug); Laodelphax striatellus Fallen (smaller brown planthopper); Leptoglossus corculus Say (leaf-footed pine seed bug); Leptodictya tabida Herrich-Schaeffer (sugarcane lace bug); Lipaphis erysimi Kaltenbach (turnip aphid); Lygocoris pabulinus Linnaeus (common green capsid); Lygus lineolaris Palisot de Beauvois (tarnished plant bug); L. Hesperus Knight (Western tarnished plant bug); L. pratensis Linnaeus (common meadow bug); L. rugulipennis Poppius (European tarnished plant bug); Macrosiphum euphorbiae Thomas (potato aphid); Macrosteles quadrilineatus Forbes (aster leafhopper); Magicicada septendecim Linnaeus (periodical cicada); Mahanarva fimbriolata Stål (sugarcane spittlebug); M. posticata Stål (little cicada of sugarcane); Melanaphis sacchari Zehntner (sugarcane aphid); Melanaspis glomerate Green (black scale); Metopolophium dirhodum Walker (rose grain aphid); Myzus persicae Sulzer (peach-potato aphid, green peach aphid); Nasonovia ribisnigri Mosley (lettuce aphid); Nephotettix cinticeps Uhler (green leafhopper); N. nigropictus Stål (rice leafhopper); Nezara viridula Linnaeus (southern green stink bug); Nilaparvata lugens Stål (brown planthopper); Nysius ericae Schilling (false chinch bug); Nysius raphanus Howard (false chinch bug); Oebalus pugnax Fabricius (rice stink bug); Oncopeltus fasciatus Dallas (large milkweed bug); Orthops campestris Linnaeus; Pemphigus spp. (root aphids and gall aphids); Peregrinus maidis Ashmead (corn planthopper); Perkinsiella saccharicida Kirkaldy (sugarcane delphacid); Phylloxera devastatrix Pergande (pecan phylloxera); Planococcus citri Risso (citrus mealybug); Plesiocoris rugicollis Fallen (apple capsid); Poecilocapsus lineatus Fabricius (four-lined plant bug); Pseudatomoscelis seriatus Reuter (cotton fleahopper); Pseudococcus spp. (other mealybug complex); Pulvinaria elongata Newstead (cottony grass scale); Pyrilla perpusilla Walker (sugarcane leafhopper); Pyrrhocoridae spp.; Quadraspidiotus perniciosus Comstock (San Jose scale); Reduviidae spp.; Rhopalosiphum maidis Fitch (corn leaf aphid); R. padi Linnaeus (bird cherry-oat aphid); Saccharicoccus sacchari Cockerell (pink sugarcane mealybug); Scaptacoris castanea Perty (brown root stink bug); Schizaphis graminum Rondani (greenbug); Sipha flava Forbes (yellow sugarcane aphid); Sitobion avenae Fabricius (English grain aphid); Sogatella furcifera Horvath (white-backed planthopper); Sogatodes oryzicola Muir (rice delphacid); Spanagonicus albofasciatus Reuter (whitemarked fleahopper); Therioaphis maculata Buckton (spotted alfalfa aphid); Tinidae spp.; Toxoptera aurantii Boyer de Fonscolombe (black citrus aphid); and T. citricida Kirkaldy (brown citrus aphid); Trialeurodes abutiloneus (bandedwinged whitefly) and T. vaporariorum Westwood (greenhouse whitefly); Trioza diospyri Ashmead (persimmon psylla); and Typhlocyba pomaria McAtee (white apple leafhopper). Also included are adults and larvae of the order Acari (mites) such as Aceria tosichella Keifer (wheat curl mite); Panonychus ulmi Koch (European red mite); Petrobia latens Müller (brown wheat mite); Steneotarsonemus bancrofti Michael (sugarcane stalk mite); spider mites and red mites in the family Tetranychidae, Oligonychus grypus Baker & Pritchard, O. indicus Hirst (sugarcane leaf mite), O. pratensis Banks (Banks grass mite), O. stickneyi McGregor (sugarcane spider mite); Tetranychus urticae Koch (two spotted spider mite); T. mcdanieli McGregor (McDaniel mite); T. cinnabarinus Boisduval (carmine spider mite); T. turkestani Ugarov & Nikolski (strawberry spider mite), flat mites in the family Tenuipalpidae, Brevipalpus lewisi McGregor (citrus flat mite); rust and bud mites in the family Eriophyidae and other foliar feeding mites and mites important in human and animal health, i.e. dust mites in the family Epidermoptidae, follicle mites in the family Demodicidae, grain mites in the family Glycyphagidae, ticks in the order Ixodidae. Ixodes scapularis Say (deer tick); I. holocyclus Neumann (Australian paralysis tick); Dermacentor variabilis Say (American dog tick); Amblyomma americanum Linnaeus (lone star tick); and scab and itch mites in the families Psoroptidae, Pyemotidae, and Sarcoptidae.

Insect pests of the order Thysanura are of interest, such as Lepisma saccharina Linnaeus (silverfish); Thermobia domestica Packard (firebrat).

Additional arthropod pests covered include: spiders in the order Araneae such as Loxosceles reclusa Gertsch & Mulaik (brown recluse spider); and the Latrodectus mactans Fabricius (black widow spider); and centipedes in the order Scutigeromorpha such as Scutigera coleoptrata Linnaeus (house centipede). In addition, insect pests of the order Isoptera are of interest, including those of the termitidae family, such as, but not limited to, Cornitermes cumulans Kollar, Cylindrotermes nordenskioeldi Holmgren and Pseudacanthotermes militaris Hagen (sugarcane termite); as well as those in the Rhinotermitidae family including, but not limited to Heterotermes tenuis Hagen. Insects of the order Thysanoptera are also of interest, including but not limited to thrips, such as Stenchaetothrips minutus van Deventer (sugarcane thrips).

Compositions comprising isolated pesticide-encoding nucleic acid molecules and pesticidal polypeptides can be identified and isolated by the methods of the invention. The compositions include nucleic acid molecules from bacterial strains having pesticide-encoding plasmids, which can be isolated and identified by the methods described herein. The compositions also include variants and fragments of the pesticide-encoding nucleic acid molecules and pesticidal polypeptides. The isolated, pesticide-encoding nucleic acid molecules can be used to create transgenic organisms such as plants that are resistant to an insect pest susceptible to the encoded pesticidal polypeptide. Likewise, the pesticidal polypeptides can be used as pesticides to control insect pests or can be used for isolating and identifying homologous pesticidal polypeptides.

Methods

As noted above, the methods involve identifying and isolating pesticidal proteins and the nucleotide sequences that encode such proteins. As a first step, the methods involve selecting or providing a bacterial strain having or suspected of having at least one pesticidal protein. It is intended that any bacterial strain having or suspected of having a pesticide-encoding nucleic acid molecule can be used in the methods described herein.

Methods of selecting a bacterial strain having or suspected of having a pesticide-encoding plasmid are well known in the art. See, e.g., de Medeiros Gitahy et al. (2007) Braz. J. Microbiol. 38:531-537; Ibarra et al. (2003) Appl. Environ. Microbiol. 69:5269-5274; Rampersad & Ammons (2005) BMC Microbiol. 5:52; and Travers et al. (1987) Appl. Environ. Microbiol. 53:1263-1266; as well as U.S. Pat. Nos. 5,573,766 and 5,997,269. Methods for selecting such a bacterial strain include, but are not limited to, insect bioassays, microscopy of a sample for pesticidal polypeptide crystals, PCR with general primers from conserved regions of nucleic acid molecules encoding the pesticidal polypeptide, etc. For example, samples from environmental sources such as sand and soil, organism sources such as nematodes, as well as plant samples, can be examined microscopically for pesticidal polypeptide crystals.

Methods of measuring pesticidal activity by insect bioassays are well known in the art. See, e.g., Brooke et al. (2001) Bull. Entomol. Res. 91:265-272; Chen et al. (2007) Proc. Natl. Acad. Sci. USA 104:13901-13906; Crespo et al. (2008) Appl. Environ. Microb. 74:130-135; Khambay et al. (2003) Pest Manag. Sci. 59:174-182; Liu & Dean (2006) Protein Eng. Des. Sel. 19:107-111; Marrone et al. (1985) J. Econ. Entomol. 78:290-293; Robertson et al., Pesticide Bioassays with Arthropods (2^{nd} ed., CRC Press 2007); Scott & McKibben (1976) J. Econ. Entomol. 71:343-344; Strickman (1985) Bull. Environ. Contam. Toxicol. 35:133-142; and Verma et al. (1982) Water Res. 16 525-529; as well as U.S. Pat. No. 6,268,181. Examples of insect bioassays include, but are not limited to, pest mortality, pest weight loss, pest repellency, pest attraction, and other behavioral and physical changes of the pest after feeding and exposure to a pesticide or pesticidal polypeptide for an appropriate length of time. General methods include addition of the pesticide, pesticidal polypeptide or an organism having the pesticidal polypeptide to the diet source in an enclosed container. See, e.g., U.S. Pat. Nos. 6,339,144 and 6,570,005.

Methods of microscopically examining a sample for pesticidal polypeptide crystals include those described by Arcas et al. (1984) Biotechnol. Lett. 6:495-500; Bernhard (2006) FEMS Microbiol. Lett. 33:261-265; Marroquin et al. (2000) Genetics 155:1693-1699, August 2000; Ryerse et al. (1990) J. Invertebr. Pathol. 56:86-90; as well as U.S. Pat. No. 4,797,279.

Methods of PCR with general primers from conserved regions of nucleic acid molecules encoding the pesticidal polypeptide are well known in the art. See, e.g., Bourque et al. (1993) Appl. Environ. Microbiol. 59:523-527; Carozzi et al. (1991) Appl. Environ. Microbiol. 57:3057-3061; Cerón et al. (1994) Appl. Environ. Microbiol. 60:353-356; Cerón et al., (1995) Appl. Environ. Microbiol. 61:3826-3831; Chak et al. (1994) Appl. Environ. Microbiol. 60:2415-2420; Gleave et al. (1993) Appl. Environ. Microbiol. 59:1683-1687; and Kalman et al. (1993) Appl. Environ. Microbiol. 59:1131-1137.

Examples of bacterial strains known or likely to have pesticide-encoding nucleic acid molecules include, but are not limited to, strains in the genus Bacillus, the genus Clostridium, the genus Enterobacter, the genus Paecilomyces, the genus Paenibacillus, the genus Photorhabdus, the genus Proteus, non-endospore-forming members of the genus Pseudomonas, the genus Serratia, and the genus Xenorhabdus. Of particular interest herein are strains in the genus Bacillus. Examples of Bacillus spp. include, but are not limited to, B. alvei, B. brevis, B. cereus, B. coagulans, B. dendrolimus, B. firmus, B. laterosporus, B. latesporus, B. megaterium, B. subtilis, B. sphaericus, B. stearothermophilus, B. sotto, B. thuringiensis, etc.

In bacteria, pesticide-encoding nucleotide sequences are typically located on plasmids, especially conjugative plasmids. Conjugative plasmids have been described in several Bacillus spp. As used herein, “plasmid” means an extra chromosomal nucleic acid molecule separate from chromosomal DNA that is capable of replicating independently from the chromosomal DNA. Plasmids typically are circular and double-stranded and can vary in size from about 1 kilobase pairs (kbp) to over 1,000 kbp. Plasmids can contain nucleic acid sequences encoding polypeptides that provide resistance to naturally occurring antibiotics or encoding polypeptides that act as toxins such as the pesticidal polypeptides of interest herein.

Of particular interest herein are δ-endotoxins of Bacillus spp., as the specific activity of δ-endotoxins is considered highly beneficial. Unlike most insecticides, the δ-endotoxins do not have a broad spectrum of activity, so they typically do not kill beneficial insects. Furthermore, the δ-endotoxins are non-toxic to mammals, including humans, domesticated animals and wildlife.

The methods of the invention include curing the bacterial strain having or suspected of having at least one pesticidal protein of the bacterial plasmid. Methods of curing plasmids from bacterial strains are well known in the art. See, e.g., Chin et al. (2005) J. Microbiol. 43:251-256; Crameri et al. (1986) J. Gen. Microbiol. 132:819-824; Heery (1989) Nuc. Acids Res. 17:10131; Spengler et al. (2006) Curr. Drug Targets 7:823-841; Molnár et al. (1978) Genetical Res. 31:197-201; and Trevors (2006) FEMS Microbiol. Lett. 32:149-157. Likewise, kits for curing plasmids from bacterial strains are commercially available, for example, from Bangalore Genei (Bangalore, India) and Plasgene Ltd. (Birmingham, United Kingdom). As used herein, “curing” means eliminating a plasmid from a bacterial strain with a concomitant loss of the phenotype conferred by the plasmid.

Because plasmids generally are stable in bacteria, they can be cured under unfavorable conditions including chemical and/or physical agents. See generally, Mirza & Hasnain (2000) Pakistan J. Biol. Sci. 3:284-288; and Trevors (1986) FEMS Microbiol. Rev. 32:149-157. Examples of chemical agents for curing plasmids include, but are not limited to, plasmid replication interrupters such as acridine orange, acriflavin, ethidium bromide, novobiocin and sodium dodecyl sulfate, and DNA synthesis inhibitors such as mitomycin C. Examples of physical agents for curing plasmids include, but are not limited to, nutrient deprivation such as thymine starvation, sub- and super-optimal temperatures, and sub- and super-optimal pHs. See, e.g., Carlton & Brown, “Gene mutations” 222-242 In: Manual of Methods for General Bacteriology (Gerhardt et al. eds., American Society for Microbiology 1981); Caro et al. (1984) Methods Microbiol. 17:97-122; Ghosh et al. (2000) FEMS Microbiol. Lett. 183:271-274; Lebrum et al. (1992) Appl. Environ. Microbiol. 51:3183-3186; Sinha (1989) FEMS Microbiol. Lett. 57:349-352; Stanisich (1988) Methods Microbiol. 21:11-48; Tolmasky et al., “Plasmids” 709-734 In: Methods for General and Molecular Microbiology (Reddy et al. eds., American Society for Microbiology 2007).

Preferably, a population of a bacterial strain of interest can be cured by culture in sub- or super-optimal temperatures. Thus, assuming that room temperature is from about 20° C. to about 25° C., sub-optimal culture temperatures suitable for curing plasmids includes temperatures below 20° C., including 19° C. down to about −70° C. or more. Likewise, super-optimal culture temperatures suitable for curing plasmids include temperatures above 25° C., including about 26° C. up to about 50° C. and higher. Generally, the super-optimal culture temperature can be about 5° C. to about 7° C. above the normal or optimal growth temperature for the bacterial strain. For example, the super-optimal culture temperature for Bacillus spp. can be about 35° C. to about 45° C., or about 40° C.

As used herein, “about” means within a statistically meaningful range of a value such as a stated concentration range, time frame, molecular weight, volume, temperature or pH. Such a range can be within an order of magnitude, typically within 20%, more typically still within 10%, and even more typically within 5% of a given value or range. The allowable variation encompassed by “about” will depend upon the particular system under study, and can be readily appreciated by one of skill in the art.

During curing, the bacteria are held at the sub- or super-optimal culture temperature for a time sufficient to cause the plasmid to be lost from subsequent generations. Such time can be for several minutes up to about several hours. Generally, the time at the sub- or super-optimal culture temperature can be for about 6 hours to about 24 hours. For example, Bacillus spp. can be cultured at about 40° C. for about 6 hours to about 24 hours.

For example, a bacterial strain having a pesticide-encoding plasmid can be incubated at a super-optimal temperature until it reaches a late log phase, at which time it can be diluted (e.g., by about 1:20) and reincubated at the elevated temperature until late log phase is reached again. At that time, serial dilutions can be prepared and plated to obtain single colonies, which can be individually tested for loss of the plasmid by an insect bioassay as described above. Likewise, single colonies can be tested for physical absence of the plasmid by, for example, gel electrophoresis. Absence of insecticidal activity in the insect bioassay or absence of the plasmid in gel electrophoresis indicates that the bacterial strain has been cured of the plasmid and therefore is a cured bacterial strain.

As discussed in greater detail below, the cured bacterial strain then can be used as a control strain (negative control) in a differential gene expression assay such as a subtractive hybridization. Nucleic acid molecules such as mRNA can be isolated from the control strain and can be used as a source of subtractor nucleic acid molecules in the subtractive hybridization. In contrast, nucleic acid molecules can be isolated from a population of the bacterial strain not cured of pesticide-encoding plasmid (e.g., wild-type or target) and can be used as a source of target nucleic acid molecules in the subtractive hybridization.

As a third step, the methods can include isolating or purifying mRNA from the control and target strains. Methods for isolating nucleic acid molecules such as mRNA are well known in the art, the most common of which is guanidinium thiocyanate-phenol-chloroform extraction. See, Bird (2005) Methods Mol. Med. 108:139-148; Chirgwin et al. (1979) Biochem. 18:5294-5299; Chomczynski & Sacchi (1987) Anal. Biochem. 162:156-159; Chomczynski & Sacchi (2006) Nat. Protoc. 1:581-585; Okayama et al. (1987) Method. Enzymol. 154:3-28; and Vogelstein & Gillespie (1979) Proc. Nat. Acad. Sci. USA 76:615-619. Kits for isolating nucleic acid molecules are commercially available, for example, from Qiagen, Inc. (Valencia, Calif.) and Applied Biosystems, Inc. (Foster City, Calif.).

Prior to isolating or purifying mRNA, the control and target bacterial strains can be grown to an appropriate stage of the bacterial life cycle in which the pesticide-encoding plasmid is expressed.

As a fourth step, the methods include enriching for plasmid-encoded mRNAs. Such methods include subtractive hybridization of the RNAs from the control and target bacterial strains. Methods of performing subtractive hybridization are well known in the art. See, e.g., Aasheim et al. (1994) BioTechniques 16:716-721; Aasheim et al. (1996) Meth. Mol. Biol. 69:115-128; Akopyants et al. (1998) Proc. Natl. Acad. Sci. 95:13108-13113; Blumberg & Belmonte (1999) Methods Mol. Biol. 97:555-574; Camerer et al. (2000) J. Biol. Chem. 275:6580-6585; Coche et al. (1994) Nucl. Acids Res. 22:1322-1323; Distler et al. (2007) Methods Mol. Med. 135:77-90; Ferreira (1999) Microbiol. 145:1967-1975; Hampson et al. (1996) Nucl. Acids Res. 24:4832-4835; Hara et al. (1991) Nuc. Acids Res. 19:7097-7104; Lambert & Williamson (1993) Nuc. Acids Res 21:775-776; Leygue et al. (1996) BioTechniques 21:1008-1012; Lönneborg et al. (1995) Genome Res. 4:S168-S176; Rodriguez & Chader (1992) Nuc. Acids Res. 20:3528; Schoen et al. (1995) Biochem. Biophys. Res. Commun. 21:181-188; Schraml et al. (1993) Trends Genet. 9:70-71; and Sharma et al. (1993) BioTechniques 15:610-611; as well as EP Patent No. 1185699 and U.S. Pat. Nos. 5,436,142 and 5,935,788. Likewise, kits for performing subtractive hybridization are commercially available, for example, from Clontech Laboratories, Inc. (Mountain View, Calif.), Invitrogen (Carlsbad, Calif.) and Milteny Biotech (Auburn, Calif.).

Methods for in silico substraction are also available in the art. In silico comparison tools are available in the art. BLAST-based webACT can be used to identify pair-wide similarity across genome sequences. Abbott et al. (2005) Bioinformatics 21:3665-3666. MUMmer rapidly aligns a complete or partially sequenced genome against a reference template. Kurtz et al. (2004) Genome Biol. 5, R12. Mauve functions as a genome-scale multiple sequence aligner. Darling et al. (2004) Genome Res. 14:1394-1403. GenomeSubtractor is an in silico substrative hybridization tool that is available on the world wide web at bioinfo-mml.sjtu.edu.en/mGS/. In this manner, the genome of the control strain and the target strain can be sequenced and in silico substration used to remove common sequences. From the remaining sequences, coding sequences can be identified, and analyzed. Pesticidal genes can be identified by comparing potential open frame regions to known pesticidal genes. Additionally, sequences can be expressed and tested for activity.

In vitro subtractive hybridization methods can also be used to isolate nucleic acid molecules such as mRNAs that differ in abundance between two nucleic acid molecule (e.g., mRNA and/or cDNA) pools (e.g., control or subtractor and target pools). Briefly, a target mRNA pool can be enriched by hybridizing it with an excess amount of a subtractor cDNA pool containing either less or no target, thus removing common nucleic acid molecules from the target nucleic acid molecules. To assist in removing hybridized nucleic acid molecules and subtractor cDNA, the nucleic acid molecules of the subtractor cDNA can be labeled, for example, by cDNA synthesis with a biotinylated primer. The nucleic acid molecules of the target mRNA pool then can be hybridized to the cDNA of the subtractor cDNA pool, and both labeled cDNA and cDNA/mRNA hybrids can be immobilized or removed via the label. Non-labeled mRNA from the target mRNA pool representing differentially expressed genes can be isolated.

Methods for in vitro substractive hybridization are known in the art. See, McSpadden and Gardener (2006) Phytopathology 96:145-154; Kim et al. (2008) J. Med. Microbiol. 57:279-286; Herrero et al. (2005) BMC Genomics 6:94; Dwyer et al. (2004) BMC Genomics 5:15; Zhu et al. (2003) Insect Biochem. Mol. Biol. 33:541-549; Miyazaki et al. (2010) FEMS Microbiol. Lett. Mar. 25 abstract; and the like.

For example, mRNA can be isolated from a control bacterial strain and from a target bacterial strain, where the control strain differs from the target strain in that the control strain has been cured of pesticide-encoding plasmids. The mRNA of the control strain can be isolated as described above and cDNA can be generated corresponding to the control mRNA by any method known in the art for reverse-transcribing and amplifying mRNA to obtain cDNA. See, e.g., Myers & Gelfand (1991) Biochem. 30:7661-7666; as well as U.S. Pat. Nos. 5,322,770; 5,310,652; 5,322,770; 5,407,800 and 6,030,814. Likewise, kits for reverse-transcribing mRNA are commercially available, for example, from Promega (Madison, Wis.) and Qiagen (Valencia, Calif.). mRNA can be removed from the resulting mRNA/cDNA duplexes by inactivation with RNase H, leaving only subtractor cDNA.

Target mRNA and subtractor cDNA can be mixed, heat-denatured for about 5 minutes to about 10 minutes at about 70° C. and cooled on ice for about 5 minutes. The mixture then can be hybridized, for example, overnight at about 68° C. under stringent conditions, although the temperature can be reduced to about 42° C. or even to room temperature to reduce the stringency. A suitable hybridization buffer can be about 0.1-2×SSC, about 0.1-2×SSPE, about 50 mM Tris-acetate pH 7.5 and about 20-300 mM NaCl. Following hybridization, the target mRNA/subtractor cDNA duplexes can be removed, leaving unique, differentially expressed target mRNA.

As used herein, “stringent conditions” means conditions under which one nucleic acid molecule (e.g., subtractor cDNA) will hybridize to its target to a detectably greater degree than to other sequences (e.g., at least two-fold over background). Stringent conditions can be sequence-dependent and will be different in different circumstances. By controlling the stringency of the hybridization and/or washing conditions, target sequences that are 100% complementary to the subtractor cDNA can be identified (i.e., homologous probing). Alternatively, the stringent condition can be adjusted to allow some mismatching in sequences so that lower degrees of similarity are detected (i.e., heterologous probing).

Typically, stringent conditions can be one in which the salt concentration is less than about 1.5 M Na⁻, typically about 0.01 to 1.0 M Na⁺ (or other salts) at about pH 7.0 to 8.3, and the temperature is at least about 30° C. for short cDNA molecules (e.g., 10 to 50 nucleotides) and at least about 60° C. for long cDNA molecules (e.g., greater than 50 nucleotides).

Stringent conditions also can be achieved with the addition of destabilizing agents such as formamide. An exemplary low stringent condition includes hybridization with a buffer solution of about 30% to about 35% formamide, 1 M NaCl, 1% SDS (sodium dodecyl sulphate) at about 37° C., and a wash in 1× to 2×SSC (20×SSC=3.0 M NaCl/0.3 M trisodium citrate) at about 50° C. to about 55° C. An exemplary moderate stringent condition includes hybridization in about 40% to about 45% formamide, 1.0 M NaCl, 1% SDS at about 37° C., and a wash in 0.5× to 1×SSC at about 55° C. to about 60° C. An exemplary high stringent condition includes hybridization in about 50% formamide, 1 M NaCl, 1% SDS at about 37° C., and a wash in 0.1×SSC at about 60° C. to about 65° C. Optionally, wash buffers may comprise about 0.1% to about 1% SDS. The duration of hybridization generally can be less than about 24 hours, usually about 4 hours to about 12 hours. The duration of the wash time can be at least a length of time sufficient to reach equilibrium.

Specificity is typically the function of post-hybridization washes, the critical factors being the ionic strength and temperature of the final wash solution. For DNA-DNA hybrids, the Tₘ can be approximated from the equation of Meinkoth & Wahl (Meinkoth & Wahl (1984) Anal. Biochem. 138:267-284; Tₘ=81.5° C.+16.6 (log M)+0.41 (%GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs). As used herein, “melting temperature” or “Tₘ” means the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at about 6° C., 7° C., 8° C., 9° C. or 10° C. lower than the thermal melting point (Tₘ); low stringency conditions can utilize a hybridization and/or wash at about 11° C., 12° C., 13° C., 14° C., 15° C. or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than about 45° C. (aqueous solution) or about 32° C. (formamide solution), it is optimal to increase the SSC concentration so that a higher temperature can be used. Methods of hybridizing nucleic acid molecules are well known in the art. See, e.g., Tijssen, Laboratory Techniques in Biochemistry and Molecular Biology—Hybridization with Nucleic Acid Probes, Part I, Chapter 2 (Elsevier 1993); and Current Protocols in Molecular Biology, Chapter 2 (Ausubel et al. eds., Greene Publishing and Wiley-Interscience 1995); and Sambrook & Russell, Molecular Cloning: A Laboratory Manual (3^{rd} ed., Cold Spring Harbor Laboratory Press 2001).

Methods for removing mRNA/cDNA duplexes include, but are not limited to, enzymatic degradation, chemical cross-linking, hydroxyapatite chromatography, or capture of duplexes with magnetic beads or monoclonal antibodies to duplexes. See, e.g., Aasheim et al. (1997) Methods Mol. Biol. 69:115-128; Clapp et al. (2007) Insect Mol. Biol. 1:133-138; Lin &Ying (2003) Methods Mol. Biol. 221:239-251; Ying & Lin (2003) Methods Mol. Biol. 221:253-259; and Lönneborg et al. (1995), supra; as well as U.S. Pat. Nos. 5,268,289 and 5,591,575.

As a fifth step, the methods can include generating a cDNA library from the unique, differentially expressed target mRNA. Methods for generating cDNA libraries from nucleic acid molecules such as mRNA are well known in the art. See, e.g., Gubler & Hoffman (1983) Gene 25:263-269; Lönneborg et al. (1995), supra; Ohara & Temple (2001) Nuc. Acids Res. 29:e22; and Okayama & Berg (1982) Mol. Cell. Biol. 2:161-170; as well as U.S. Pat. Nos. 5,512,468; 5,525,486 and 5,707,841. Kits for generating cDNA libraries are commercially available, for example, from Clontech, Dualsystems Biotech AG (Schlieren, Switzerland), GE Healthcare Bio-Sciences Corp. (Piscataway, N.J.), Invitrogen and Stratagene (La Jolla, Calif.).

As used herein, “cDNA library” means a collection of cloned cDNA molecules inserted into a collection of host cells such as bacteria, which together constitute some portion of the transcriptome of the target strain. The host cell therefore is a self-replicating organism that can be used to maintain a library or a piece of cDNA of interest. Because cDNA is produced from fully transcribed mRNA found in the target strain, the cDNA library therefore contains only the differentially expressed nucleic acid molecules of the target strain, such as pesticide-encoding plasmids.

It has been believed that bacterial mRNA typically is not polyadenylated or lack the relatively stable poly(A) tails found on eukaryotic messages. However, it has been discovered that the target mRNA molecules specifically those encoding pesticidal proteins are polyadenylated transcripts. Thus, in one embodiment, oligo(dT) primers can be used to isolate and/or amplify the mRNA. See, Weiss et al. (1976) J. Biol. Chem. 251:3425-3431; Verma, I. M. (1978) J. Virol. 26:615-629; and Hagenburchle et al. (1979) J. Biol. Chem. 254:7157-7162. Further, oligo(dT) primers and kits are commercially available. The discovery that this bacterial mRNA can be used as a template with oligo(dT) primers aids in many molecular techniques and has been used to amplify the mRNA via reverse transcription, cDNA synthesis and T7 transcription. The polyA tails on the transcripts are also useful for creating cDNA libraries of the target mRNAs. Since the bacterial mRNA may be naturally polyadenylated, isolation, purification and reverse-transcription of mRNA from either the control strain or target strain can be aided by, for example, poly(T) resins for isolation/purification or 5′ biotinylated oligo(dT) primers for reverse-transcription reactions.

The cDNA can be ligated into a vector to allow introduction of the cDNA into the appropriate host cells. As used herein, “vector” means a replicon, such as a plasmid, phage or cosmid, to which another nucleic acid segment may be attached so as to bring about the replication of the attached segment. A vector is capable of transferring nucleic acid molecules to the host cells. Bacterial vectors typically can be of plasmid or phage origin.

For example, the unique, differentially expressed target mRNA described above can be added to a microcentrifuge tube having a vector such as bacterial plasmid (e.g., pCRII backbone; Invitrogen), ligase buffer, ATP, water and a DNA ligase such as a T4 ligase. For example, the mixture of mRNA and plasmid can be incubated overnight at about 16° C. The mixture can be heated to about 75° C. for about 10 minutes to heat-inactivate the ligase. The mixture then can be added to competent host cells to transform the host cells. The host cells can be prokaryotic cells, especially various strains of Escherichia coli or Bacillus; however, other bacterial strains can be used.

Restriction enzymes can be used to introduce cuts into the target mRNA and the plasmid to facilitate insertion of the target mRNA into the plasmid. Moreover, restriction enzyme adapters such as EcoRI/NotI adapters can be added to the target mRNA when the desired restriction enzyme sites are not present within it. Methods of adding restriction enzyme adapters are well known in the art. See, e.g., Krebs et al. (2006) Anal. Biochem. 350:313-315; and Lönneborg et al. (1995), supra. Likewise, kits for adding restriction enzyme sites are commercially available, for example, from Invitrogen.

Alternatively, viruses such as bacteriophages can be used as the vector to deliver the target mRNA to competent host cells. Vectors can be constructed using standard molecular biology techniques as described, for example, in Sambrook & Russell (2001), supra.

As a sixth step, the methods can include screening the cDNA library for pesticide-encoding nucleotide sequences or pesticidal polypeptides. Methods of screening cDNA libraries for nucleic acid sequences are well known in the art. See, e.g., Munroe et al. (1995) Proc. Natl. Acad. Sci. USA 92:2209-2213; Sambrook & Russell (2001), supra; and Takumi (1997) Methods Mol. Biol. 67:339-344.

Briefly, the cDNA library can be screened by initially performing PCR using plasmid DNA as template. Alternatively, primers and probes from known Cry toxins can be used in the methods described herein to identify homologous or similar pesticide-encoding nucleic acid molecules in the cDNA library. Pesticidal polypeptides such as δ-endotoxins generally have five conserved sequence domains, and three conserved structural domains (see, e.g., de Maagd et al. (2001) Trends Genetics 17:193-199). The first conserved structural domain (Domain I) consists of seven alpha helices and is involved in membrane insertion and pore formation. The second conserved structural domain (Domain II) consists of three beta-sheets arranged in a Greek key configuration, and the third conserved structural domain (Domain III) consists of two antiparallel beta-sheets in “jelly-roll” formation. Domains II and III are involved in receptor recognition and binding, and are therefore considered determinants of toxin specificity. A list of known δ-endotoxins (Cry and Cyt endotoxins) and their GenBank® Accession Nos. are listed in Table 1, which can be used as a source for nucleic and amino acid sequences for primers, probes, etc.

TABLE 1
Known δ-endotoxins and their GenBank ® Accession Nos.

| Endotoxin | GenBank ® Accession No. |
|---|---|
| Cry1Aa1 | AAA22353 |
| Cry1Aa2 | AAA22552 |
| Cry1Aa3 | BAA00257 |
| Cry1Aa4 | CAA31886 |
| Cry1Aa5 | BAA04468 |
| Cry1Aa6 | AAA86265 |
| Cry1Aa7 | AAD46139 |
| Cry1Aa8 | I26149 |
| Cry1Aa9 | BAA77213 |
| Cry1Aa10 | AAD55382 |
| Cry1Aa11 | CAA70856 |
| Cry1Aa12 | AAP80146 |
| Cry1Aa13 | AAM44305 |
| Cry1Aa14 | AAP40639 |
| Cry1Aa15 | AAY66993 |
| Cry1Ab1 | AAA22330 |
| Cry1Ab2 | AAA22613 |
| Cry1Ab3 | AAA22561 |
| Cry1Ab4 | BAA00071 |
| Cry1Ab5 | CAA28405 |
| Cry1Ab6 | AAA22420 |
| Cry1Ab7 | CAA31620 |
| Cry1Ab8 | AAA22551 |
| Cry1Ab9 | CAA38701 |
| Cry1Ab10 | A29125 |
| Cry1Ab11 | I12419 |
| Cry1Ab12 | AAC64003 |
| Cry1Ab13 | AAN76494 |
| Cry1Ab14 | AAG16877 |
| Cry1Ab15 | AAO13302 |
| Cry1Ab16 | AAK55546 |
| Cry1Ab17 | AAT46415 |
| Cry1Ab18 | AAQ88259 |
| Cry1Ab19 | AAW31761 |
| Cry1Ab20 | ABB72460 |
| Cry1Ab21 | ABS18384 |
| Cry1Ab22 | ABW87320 |
| Cry1Ab-like | AAK14336 |
| Cry1Ab-like | AAK14337 |
| Cry1Ab-like | AAK14338 |
| Cry1Ab-like | ABG88858 |
| Cry1Ac1 | AAA22331 |
| Cry1Ac2 | AAA22338 |
| Cry1Ac3 | CAA38098 |
| Cry1Ac4 | AAA73077 |
| Cry1Ac5 | AAA22339 |
| Cry1Ac6 | AAA86266 |
| Cry1Ac7 | AAB46989 |
| Cry1Ac8 | AAC44841 |
| Cry1Ac9 | AAB49768 |
| Cry1Ac10 | CAA05505 |
| Cry1Ac11 | CAA10270 |
| Cry1Ac12 | I12418 |
| Cry1Ac13 | AAD38701 |
| Cry1Ac14 | AAQ06607 |
| Cry1Ac15 | AAN07788 |
| Cry1Ac16 | AAU87037 |
| Cry1Ac17 | AAX18704 |
| Cry1Ac18 | AAY88347 |
| Cry1Ac19 | ABD37053 |
| Cry1Ac20 | ABB89046 |
| Cry1Ac21 | AAY66992 |
| Cry1Ac22 | ABZ01836 |
| Cry1Ac23 | CAQ30431 |
| Cry1Ac24 | ABL01535 |
| Cry1Ac25 | FJ513324 |
| Cry1Ac26 | FJ617446 |
| Cry1Ac27 | FJ617447 |
| Cry1Ac28 | ACM90319 |
| Cry1Ad1 | AAA22340 |
| Cry1Ad2 | CAA01880 |
| Cry1Ae1 | AAA22410 |
| Cry1Af1 | AAB82749 |
| Cry1Ag1 | AAD46137 |
| Cry1Ah1 | AAQ14326 |
| Cry1Ah2 | ABB76664 |
| Cry1Ai1 | AAO39719 |
| Cry1A-like | AAK14339 |
| Cry1Ba1 | CAA29898 |
| Cry1Ba2 | CAA65003 |
| Cry1Ba3 | AAK63251 |
| Cry1Ba4 | AAK51084 |
| Cry1Ba5 | ABO20894 |
| Cry1Ba6 | ABL60921 |
| Cry1Bb1 | AAA22344 |
| Cry1Bc1 | CAA86568 |
| Cry1Bd1 | AAD10292 |
| Cry1Bd2 | AAM93496 |
| Cry1Be1 | AAC32850 |
| Cry1Be2 | AAQ52387 |
| Cry1Be3 | FJ716102 |
| Cry1Bf1 | CAC50778 |
| Cry1Bf2 | AAQ52380 |
| Cry1Bg1 | AAO39720 |
| Cry1Ca1 | CAA30396 |
| Cry1Ca2 | CAA31951 |
| Cry1Ca3 | AAA22343 |
| Cry1Ca4 | CAA01886 |
| Cry1Ca5 | CAA65457 |
| Cry1Ca6 [1] | AAF37224 |
| Cry1Ca7 | AAG50438 |
| Cry1Cab | AAM00264 |
| Cry1Ca9 | AAL79362 |
| Cry1Ca10 | AAN16462 |
| Cry1Ca11 | AAX53094 |
| Cry1Cb1 | M97880 |
| Cry1Cb2 | AAG35409 |
| Cry1Cb3 | ACD50894 |
| Cry1Cb-like | AAX63901 |
| Cry1Da1 | CAA38099 |
| Cry1Da2 | I76415 |
| Cry1Db1 | CAA80234 |
| Cry1Db2 | AAK48937 |
| Cry1Dc1 | ABK35074 |
| Cry1Ea1 | CAA37933 |
| Cry1Ea2 | CAA39609 |
| Cry1Ea3 | AAA22345 |
| Cry1Ea4 | AAD04732 |
| Cry1Ea5 | A15535 |
| Cry1Ea6 | AAL50330 |
| Cry1Ea7 | AAW72936 |
| Cry1Ea8 | ABX11258 |
| Cry1Eb1 | AAA22346 |
| Cry1Fa1 | AAA22348 |
| Cry1Fa2 | AAA22347 |
| Cry1Fb1 | CAA80235 |
| Cry1Fb2 | BAA25298 |
| Cry1Fb3 | AAF21767 |
| Cry1Fb4 | AAC10641 |
| Cry1Fb5 | AAO13295 |
| Cry1Fb6 | ACD50892 |
| Cry1Fb7 | ACD50893 |
| Cry1Ga1 | CAA80233 |
| Cry1Ga2 | CAA70506 |
| Cry1Gb1 | AAD10291 |
| Cry1Gb2 | AAO13756 |
| Cry1Gc | AAQ52381 |
| Cry1Ha1 | CAA80236 |
| Cry1Hb1 | AAA79694 |
| Cry1H-like | AAF01213 |
| Cry1Ia1 | CAA44633 |
| Cry1Ia2 | AAA22354 |
| Cry1Ia3 | AAC36999 |
| Cry1Ia4 | AAB00958 |
| Cry1Ia5 | CAA70124 |
| Cry1Ia6 | AAC26910 |
| Cry1Ia7 | AAM73516 |
| Cry1Ia8 | AAK66742 |
| Cry1Ia9 | AAQ08616 |
| Cry1Ia10 | AAP86782 |
| Cry1Ia11 | CAC85964 |
| Cry1Ia12 | AAV53390 |
| Cry1Ia13 | ABF83202 |
| Cry1Ia14 | ACG63871 |
| Cry1Ia15 | FJ617445 |
| Cry1Ia16 | FJ617448 |
| Cry1Ib1 | AAA82114 |
| Cry1Ib2 | ABW88019 |
| Cry1Ib3 | ACD75515 |
| Cry1Ic1 | AAC62933 |
| Cry1Ic2 | AAE71691 |
| Cry1Id1 | AAD44366 |
| Cry1Ie1 | AAG43526 |
| Cry1If1 | AAQ52382 |
| Cry1I-like | AAC31094 |
| Cry1I-like | ABG88859 |
| Cry1Ja1 | AAA22341 |
| Cry1Jb1 | AAA98959 |
| Cry1Jc1 | AAC31092 |
| Cry1Jc2 | AAQ52372 |
| Cry1Jd1 | CAC50779 |
| Cry1Ka1 | AAB00376 |
| Cry1La1 | AAS60191 |
| Cry1-like | AAC31091 |
| Cry2Aa1 | AAA22335 |
| Cry2Aa2 | AAA83516 |
| Cry2Aa3 | D86064 |
| Cry2Aa4 | AAC04867 |
| Cry2Aa5 | CAA10671 |
| Cry2Aa6 | CAA10672 |
| Cry2Aa7 | CAA10670 |
| Cry2Aa8 | AAO13734 |
| Cry2Aa9 | AAO13750 |
| Cry2Aa10 | AAQ04263 |
| Cry2Aa11 | AAQ52384 |
| Cry2Aa12 | ABI83671 |
| Cry2Aa13 | ABL01536 |
| Cry2Aa14 | ACF04939 |
| Cry2Ab1 | AAA22342 |
| Cry2Ab2 | CAA39075 |
| Cry2Ab3 | AAG36762 |
| Cry2Ab4 | AAO13296 |
| Cry2Ab5 | AAQ04609 |
| Cry2Ab6 | AAP59457 |
| Cry2Ab7 | AAZ66347 |
| Cry2Ab8 | ABC95996 |
| Cry2Ab9 | ABC74968 |
| Cry2Ab10 | EF157306 |
| Cry2Ab11 | CAM84575 |
| Cry2Ab12 | ABM21764 |
| Cry2Ab13 | ACG76120 |
| Cry2Ab14 | ACG76121 |
| Cry2Ac1 | CAA40536 |
| Cry2Ac2 | AAG35410 |
| Cry2Ac3 | AAQ52385 |
| Cry2Ac4 | ABC95997 |
| Cry2Ac5 | ABC74969 |
| Cry2Ac6 | ABC74793 |
| Cry2Ac7 | CAL18690 |
| Cry2Ac8 | CAM09325 |
| Cry2Ac9 | CAM09326 |
| Cry2Ac10 | ABN15104 |
| Cry2Ac11 | CAM83895 |
| Cry2Ac12 | CAM83896 |
| Cry2Ad1 | AAF09583 |
| Cry2Ad2 | ABC86927 |
| Cry2Ad3 | CAK29504 |
| Cry2Ad4 | CAM32331 |
| Cry2Ad5 | CAO78739 |
| Cry2Ae1 | AAQ52362 |
| Cry2Af1 | ABO30519 |
| Cry2Ag | ACH91610 |
| Cry2Ah | EU939453 |
| Cry2Ah2 | ACL80665 |
| Cry2Ai | FJ788388 |
| Cry3Aa1 | AAA22336 |
| Cry3Aa2 | AAA22541 |
| Cry3Aa3 | CAA68482 |
| Cry3Aa4 | AAA22542 |
| Cry3Aa5 | AAA50255 |
| Cry3Aa6 | AAC43266 |
| Cry3Aa7 | CAB41411 |
| Cry3Aa8 | AAS79487 |
| Cry3Aa9 | AAW05659 |
| Cry3Aa10 | AAU29411 |
| Cry3Aa11 | AAW82872 |
| Cry3Aa12 | ABY49136 |
| Cry3Ba1 | CAA34983 |
| Cry3Ba2 | CAA00645 |
| Cry3Bb1 | AAA22334 |
| Cry3Bb2 | AAA74198 |
| Cry3Bb3 | I15475 |
| Cry3Ca1 | CAA42469 |
| Cry4Aa1 | CAA68485 |
| Cry4Aa2 | BAA00179 |
| Cry4Aa3 | CAD30148 |
| Cry4A-like | AAY96321 |
| Cry4Ba1 | CAA30312 |
| Cry4Ba2 | CAA30114 |
| Cry4Ba3 | AAA22337 |
| Cry4Ba4 | BAA00178 |
| Cry4Ba5 | CAD30095 |
| Cry4Ba-like | ABC47686 |
| Cry4Ca1 | EU646202 |
| Cry4Cb1 | FJ403208 |
| Cry4Cb2 | FJ597622 |
| Cry4Cc1 | FJ403207 |
| Cry5Aa1 | AAA67694 |
| Cry5Ab1 | AAA67693 |
| Cry5Ac1 | I34543 |
| Cry5Ad1 | ABQ82087 |
| Cry5Ba1 | AAA68598 |
| Cry5Ba2 | ABW88932 |
| Cry6Aa1 | AAA22357 |
| Cry6Aa2 | AAM46849 |
| Cry6Aa3 | ABH03377 |
| Cry6Ba1 | AAA22358 |
| Cry7Aa1 | AAA22351 |
| Cry7Ab1 | AAA21120 |
| Cry7Ab2 | AAA21121 |
| Cry7Ab3 | ABX24522 |
| Cry7Ab4 | EU380678 |
| Cry7Ab5 | ABX79555 |
| Cry7Ab6 | ACI44005 |
| Cry7Ab7 | FJ940776 |
| Cry7Ab8 | GU145299 |
| Cry7Ba1 | ABB70817 |
| Cry7Ca1 | ABR67863 |
| Cry7Da1 | ACQ99547 |
| Cry8Aa1 | AAA21117 |
| Cry8Ab1 | EU044830 |
| Cry8Ba1 | AAA21118 |
| Cry8Bb1 | CAD57542 |
| Cry8Bc1 | CAD57543 |
| Cry8Ca1 | AAA21119 |
| Cry8Ca2 | AAR98783 |
| Cry8Ca3 | EU625349 |
| Cry8Da1 | BAC07226 |
| Cry8Da2 | BD133574 |
| Cry8Da3 | BD133575 |
| Cry8Db1 | BAF93483 |
| Cry8Ea1 | AAQ73470 |
| Cry8Ea2 | EU047597 |
| Cry8Fa1 | AAT48690 |
| Cry8Ga1 | AAT46073 |
| Cry8Ga2 | ABC42043 |
| Cry8Ga3 | FJ198072 |
| Cry8Ha1 | EF465532 |
| Cry8Ia1 | EU381044 |
| Cry8Ja1 | EU625348 |
| Cry8Ka1 | FJ422558 |
| Cry8Ka2 | ACN87262 |
| Cry8-like | FJ770571 |
| Cry8-like | ABS53003 |
| Cry9Aa1 | CAA41122 |
| Cry9Aa2 | CAA41425 |
| Cry9Aa3 | GQ249293 |
| Cry9Aa4 | GQ249294 |
| Cry9Aa like | AAQ52376 |
| Cry9Ba1 | CAA52927 |
| Cry9Bb1 | AAV28716 |
| Cry9Ca1 | CAA85764 |
| Cry9Ca2 | AAQ52375 |
| Cry9Da1 | BAA19948 |
| Cry9Da2 | AAB97923 |
| Cry9Da3 | GQ249295 |
| Cry9Da4 | GQ249297 |
| Cry9Db1 | AAX78439 |
| Cry9Ea1 | BAA34908 |
| Cry9Ea2 | AAO12908 |
| Cry9Ea3 | ABM21765 |
| Cry9Ea4 | ACE88267 |
| Cry9Ea5 | ACF04743 |
| Cry9Ea6 | ACG63872 |
| Cry9Ea7 | FJ380927 |
| Cry9Ea8 | GQ249292 |
| Cry9Eb1 | CAC50780 |
| Cry9Eb2 | GQ249298 |
| Cry9Ec1 | AAC63366 |
| Cry9Ed1 | AAX78440 |
| Cry9Ee1 | GQ249296 |
| Cry9-like | AAC63366 |
| Cry10Aa1 | AAA22614 |
| Cry10Aa2 | E00614 |
| Cry10Aa3 | CAD30098 |
| Cry10A-like | DQ167578 |
| Cry11Aa1 | AAA22352 |
| Cry11Aa2 | AAA22611 |
| Cry11Aa3 | CAD30081 |
| Cry11Aa-like | DQ166531 |
| Cry11Ba1 | CAA60504 |
| Cry11Bb1 | AAC97162 |
| Cry12Aa1 | AAA22355 |
| Cry13Aa1 | AAA22356 |
| Cry14Aa1 | AAA21516 |
| Cry15Aa1 | AAA22333 |
| Cry16Aa1 | CAA63860 |
| Cry17Aa1 | CAA67841 |
| Cry18Aa1 | CAA67506 |
| Cry18Ba1 | AAF89667 |
| Cry18Ca1 | AAF89668 |
| Cry19Aa1 | CAA68875 |
| Cry19Ba1 | BAA32397 |
| Cry20Aa1 | AAB93476 |
| Cry20Ba1 | ACS93601 |
| Cry20-like | GQ144333 |
| Cry21Aa1 | I32932 |
| Cry21Aa2 | I66477 |
| Cry21Ba1 | BAC06484 |
| Cry22Aa1 | I34547 |
| Cry22Aa2 | CAD43579 |
| Cry22Aa3 | ACD93211 |
| Cry22Ab1 | AAK50456 |
| Cry22Ab2 | CAD43577 |
| Cry22Ba1 | CAD43578 |
| Cry23Aa1 | AAF76375 |
| Cry24Aa1 | AAC61891 |
| Cry24Ba1 | BAD32657 |
| Cry24Ca1 | CAJ43600 |
| Cry25Aa1 | AAC61892 |
| Cry26Aa1 | AAD25075 |
| Cry27Aa1 | BAA82796 |
| Cry28Aa1 | AAD24189 |
| Cry28Aa2 | AAG00235 |
| Cry29Aa1 | CAC80985 |
| Cry30Aa1 | CAC80986 |
| Cry30Ba1 | BAD00052 |
| Cry30Ca1 | BAD67157 |
| Cry30Ca2 | ACU24781 |
| Cry30Da1 | EF095955 |
| Cry30Db1 | BAE80088 |
| Cry30Ea1 | ACC95445 |
| Cry30Ea2 | FJ499389 |
| Cry30Fa1 | ACI22625 |
| Cry30Ga1 | ACG60020 |
| Cry31Aa1 | BAB11757 |
| Cry31Aa2 | AAL87458 |
| Ciy31Aa3 | BAE79808 |
| Cry31Aa4 | BAF32571 |
| Cry31Aa5 | BAF32572 |
| Cry31Ab1 | BAE79809 |
| Cry31Ab2 | BAF32570 |
| Cry31Ac1 | BAF34368 |
| Cry32Aa1 | AAG36711 |
| Cry32Ba1 | BAB7860I |
| Cry32Ca1 | BAB78602 |
| Cry32Da1 | BAB78603 |
| Cry33Aa1 | AAL26871 |
| Cry34Aa1 | AAG50341 |
| Cry34Aa2 | AAK64560 |
| Cry34Aa3 | AAT29032 |
| Cry34Aa4 | AAT29030 |
| Cry34Ab1 | AAG41671 |
| Cry34Ac1 | AAG50118 |
| Cry34Ac2 | AAK64562 |
| Cry34Ac3 | AAT29029 |
| Cry34Ba1 | AAK64565 |
| Cry34Ba2 | AAT29033 |
| Cry34Ba3 | AAT29031 |
| Cry35Aa1 | AAG50342 |
| Cry35Aa2 | AAK64561 |
| Cry35Aa3 | AAT29028 |
| Cry35Aa4 | AAT29025 |
| Cry35Ab1 | AAG41672 |
| Cry35Ab2 | AAK64563 |
| Cry35Ab3 | AY536891 |
| Cry35Ac1 | AAG50117 |
| Cry35Ba1 | AAK64566 |
| Cry35Ba2 | AAT29027 |
| Cry35Ba3 | AAT29026 |
| Cry36Aa1 | AAK64558 |
| Cry37Aa1 | AAF76376 |
| Cry38Aa1 | AAK64559 |
| Cry39Aa1 | BAB72016 |
| Cry40Aa1 | BAB72018 |
| Cry40Ba1 | BAC77648 |
| Cry40Ca1 | EU381045 |
| Cry40Da1 | ACF15199 |
| Cry41Aa1 | BAD35157 |
| Cry41Ab1 | BAD35163 |
| Cry42Aa1 | BAD35166 |
| Cry43Aa1 | BAD15301 |
| Cry43Aa2 | BAD95474 |
| Cry43Ba1 | BAD15303 |
| Cry43-like | BAD15305 |
| Cry44Aa | BAD08532 |
| Cry45Aa | BAD22577 |
| Cry46Aa | BAC79010 |
| Cry46Aa2 | BAG68906 |
| Cry46Ab | BAD35170 |
| Cry47Aa | AAY24695 |
| Cry48Aa | CAJ18351 |
| Cry48Aa2 | CAJ86545 |
| Cry48Aa3 | CAJ86546 |
| Cry48Ab | CAJ86548 |
| Cry48Ab2 | CAJ86549 |
| Cry49Aa | CAH56541 |
| Cry49Aa2 | CAJ86541 |
| Cry49Aa3 | CAJ86543 |
| Cry49Aa4 | CAJ86544 |
| Cry49Ab1 | CAJ86542 |
| Cry50Aa1 | BAE86999 |
| Cry51Aa1 | ABI14444 |
| Cry52Aa1 | EF613489 |
| Cry52Ba1 | FJ361760 |
| Cry53Aa1 | EF633476 |
| Cry53Ab1 | FJ361759 |
| Cry54Aa1 | ACA52194 |
| Cry55Aa1 | ABW88931 |
| Cry55Aa2 | AAE33526 |
| Cry56Aa1 | FJ597621 |
| Cry56Aa2 | GQ483512 |
| Cry57Aa1 | ANC87261 |
| Cry58Aa1 | ANC87260 |
| Cry59Aa1 | ACR43758 |
| Cyt1Aa1 | X03182 |
| Cyt1Aa2 | X04338 |
| Cyt1Aa3 | Y00135 |
| Cyt1Aa4 | M35968 |
| Cyt1Aa5 | AL731825 |
| Cyt1Aa6 | ABC17640 |
| Cyt1Aa-like | ABB01172 |
| Cyt1Ab1 | X98793 |
| Cyt1Ba1 | U37196 |
| Cyt1Ca1 | AL731825 |
| Cyt2Aa1 | Z14147 |
| Cyt2Aa2 | AF472606 |
| Cyt2Aa3 | EU835185 |
| Cyt2Ba1 | U52043 |
| Cyt2Ba2 | AF020789 |
| Cyt2Ba3 | AF022884 |
| Cyt2Ba4 | AF022885 |
| Cyt2Ba5 | AF022886 |
| Cyt2Ba6 | AF034926 |
| Cyt2Ba7 | AF215645 |
| Cyt2Ba8 | AF215646 |
| Cyt2Ba9 | AL731825 |
| Cyt2Ba10 | ACX54358 |
| Cyt2Ba11 | ACX54359 |
| Cyt2Ba12 | ACX54360 |
| Cyt2Ba-like | ABE99695 |
| Cyt2Bb1 | U82519 |
| Cyt2Bc1 | CAC80987 |
| Cyt2B-like | DQ341380 |
| Cyt2Ca1 | AAK50455 |
| Unknown | AAA22332 |
| Unknown | AAL26870 |
| Unknown | CAA63374 |
| Unknown | BAA13073 |
| Unknown | CAA67205 |
| Unknown | CAA67329 |

Because the pesticidal polypeptides of interest herein typically have a distinct crystal structure, the cDNA library also can be screened microscopically. Alternatively, the cDNA library can be screened with antibodies to known pesticidal polypeptides. As noted above antibodies to Cry toxins are well known in the art and are commercially available.

Clones identified as having a pesticide-encoding nucleic acid molecule or pesticidal polypeptide by any of the above screens then can be subjected to further analysis such as amino or nucleic acid sequencing.

As a seventh step, the methods can include sequencing the pesticide-encoding nucleic acid molecule or sequencing the pesticidal polypeptide isolated in the cDNA library screen. Methods of sequencing nucleic acid molecules are well known in the art. See, e.g., Edwards et al. (2005) Mut. Res. 573:3-12; Hanna et al. (2000) J. Clin. Microbiol. 38:2715-2721; Ju et al. (1995) Proc. Natl. Acad. Sci. USA 92:4347-4351; Maxam & Gilbert (1977) Proc. Natl. Acad. Sci. USA 74:560-564; Ramanathan et al. (2004) Anal. Biochem. 330:227-241; Ronaghi et al. (1996) Anal. Biochem. 242:84-89; Sanger et al. (1977) Proc. Natl. Acad. Sci. USA 74:5463-5467; and Smith et al. (1986) Nature 321:674-679; as well as U.S. Pat. Nos. 5,750,341 and 5,795,782.

Methods of sequencing polypeptides also are well known in the art. See, e.g., Edman (1950) Acta Chem. Scand. 4:283; Henzel et al. (1993) Proc. Natl. Acad. Sci. USA 90:5011-5015; James et al. (1993) Biochem. Biophys. Res. Commun. 195:58-64; Liu et al. (1983) Int. J. Pept. Protein Res. 21:209-215; Mann et al. (1993) Biol. Mass Spectrom. 22:338-345; Niall (1973) Meth. Enzymol. 27:942-1010; Oike et al. (1982) J. Biol. Chem. 257:9751-9758; Pappin et al. (1993) Curr. Biol. 3:327-332; Steen & Mann (2004) Nat. Rev. Mol. Cell Biol. 5:699-711; and Yates et al. (1993) Anal. Biochem. 214:397-408.

As used herein, “sequencing” means determining the primary structure (or primary sequence) of an unbranched biopolymer such as determining the nucleotide sequence of a nucleic acid molecule or the amino acid sequence of a polypeptide.

As such, a pesticide-encoding nucleic acid molecule or pesticidal polypeptide can be identified by being sequenced. For example, the obtained nucleotide or amino acid sequence can be compared to sequences from known pesticidal polypeptides, such as those listed above in Table 1. If the pesticidal polypeptide is found to be new, its pesticidal activity and pest specificity can be determined by performing insect bioassays as described above against any of the insect pests described above.

Identified polypeptides exhibiting activity against an insect pest of interest can be used in pesticide formulations such as dusts, solids and sprays. The pesticidal formulations and can be applied to the crop area or plant to be treated, simultaneously or in succession, with other compounds. Nucleic acid molecules encoding the pesticidal polypeptides can be used in DNA constructs for expression in plants or plant cells.

Unless defined otherwise, all technical and scientific terms used herein have the same meaning as commonly understood by one of skill in the art to which the invention pertains. Although any methods and materials similar to or equivalent to those described herein can be used in the practice or testing of the present invention, the preferred methods and materials are described herein.

The following examples are offered by way of illustration and not by way of limitation.

### EXPERIMENTAL

### EXAMPLE 1

### Bioinformatic Subtraction Via Solexa Sequencing

1. cDNA library sequencing of cured/normalizer strain or utilize Bacillus genome databases
2. Sequencing of experimental cDNA from active insecticidal protein strain
3. Electronic subtraction of share sequences, leaving novel sequences
4. Analyze the novel sequences for putative insecticidal genes
   Adapted step #1: utilized a different strain with a totally different spectrum of insecticidal activity. Example: a Lepidopteran strain was used to subtract from a Coleopteran active strain, and vice versa.

### EXAMPLE 2

### General Protocol for RNA preparation for Transcript Analysis Approach to Novel Gene Discovery

1. Identify a strain with bioactivity of interest, in this case bacteria, in particular Bt.
2. Culture strain in the medium which produces the bioactive proteins. In the case of Bt, we identified that the proteins expressed in the crystal portion have good transcript numbers around 16-26 hours, depending on the strain and the media used.
3. Harvest strain following Qiagen's RNAProtect Bacteria Protocol.
4. Prep total RNA following Qiagen's RNAProtect Bacteria Protocol, performing on Column DNase digestion.
5. After total RNA isolation from the strain, the RNA was DNase treated again, using Ambion's TURBO DNA-free kit.
6. QC was performed using Agilent's Bioanalyzer Nano6000 RNA Chip.
   a. If high quality RNA is obtained, as indicated by the RIN and the 16s/23s ratios, the RNA is moved through the rest of the protocol
7. Ambion's MICROBExpress kit is used to remove the majority of rRNA from the total RNA. The protocol is performed at least once and may be performed multiple times.
8. After QC, the RNA still showed quite a bit of smaller RNAs, so it was run through Qiagen's RNA Cleanup protocol to help removed RNA <200 nt.
9. The mRNA was run through another Nano6000 chip for QC and concentration determination.
10. The mRNA was then submitted for Solexa sequencing. Two different strains were submitted at the same time with different spectrums of bioactivity
11. The sequence was used to subtract the two data sets, in silico, producing a pool of data left with only the novel sequences in each strain. A novel Cry22B-like polypeptide was identified in one of the strains (BD574). Once the data was masked, novel sequences were identified that appear to be of interest and that may be responsible for the insecticidal bioactivity.
   The technique can be applied to more species of bacteria and even other active samples.

### EXAMPLE 3

### Preparation of Bacterial RNA for Solexa Sequence analysis (Transcript Analysis)

Bacterial strains of interest, identified as having bioactivity against one or multiple target insects, were grown in the appropriate media in which the bacterium produces the bioactive molecule. Overnight cultures in terrific broth are inoculated from a pure culture glycerol stock or a single colony and then they are grown overnight at 30° C. with agitation. The following day, the media used to express the bioactive molecule is inoculated in a baffled flask using 1/100th of the overnight culture and incubated at 30° C. and 275-300 rpm. After 18-26 hours of growth, the cultures are examined microscopically to observe the stage of the bacterium, in particular the degree of sporulation for Bacillus thuringiensis strains. Various time points are generally harvested as the cells continue through their various stages, following Qiagen's RNAprotect Bacterial reagent's protocol 4, using 20 mg/ml lysozyme and incubating with constant vortexing for approximately 1 hour. Total RNA purification following Qiagen's RNeasy Mini (Protocol 7) or Midi (Protocol 8) is then performed utilizing the optional on-column DNase digestion.

Total RNA quality is determined utilizing an Agilent Bioanalyzer and the RNA Nano6000 chip system, following Agilent's protocols. Total RNA passing QC is moved onto the stage where the rRNA is removed using Ambion's MICROBExpress kit with the following modifications: at step B.4., the incubation time was increased up to 1 hour and in step E.2.a., only 15-20 μl of nuclease-free water is used to resuspend the enriched mRNA. The enriched mRNA is run on a Bioanalyzer Nano6000 chip using the mRNA assay option. If sufficient rRNA has been removed and the concentration is enough to submit for Solexa transcript sequencing, it is then provided to the group performing the Solexa analysis. If sufficient rRNA has not been removed, the MICROBExpress protocol is repeated, as well as the QC on the Bioanalyzer. An RNA Clean-up can be performed using Qiagen's RNeasy Mini column and protocol. The RNA is then precipitated and resuspended in an appropriate volume of nuclease-free water.

At least 100 ng of enriched mRNA is provided for Solexa sequencing and prepared following the appropriate manufacturer's (Illumina) protocols.

### EXAMPLE 4

### Curing Procedure

The strain of interest was grown in LB media at 42° C. with shaking overnight and a portion of the overnight culture was used to inoculate a new culture of the strain to be grown at 42° C. This process was repeated 4 to 11 times. A portion of the culture grown at 42° C. was then plated on LB agar plates and allowed to grow overnight at 30° C. Single colonies were isolated and cultured in media which produced the proteins of interest. These samples were tested in an insect bioassay. From the colonies that demonstrated a lack of activity, Colony PCR was performed on known genes within the strains. For example, in strain DP1019, which contains Cry9Db1, PCR was performed to see if the gene was present. The uncured strain was used as a control for both bioassay and colony PCR. The cultures were also examined under the microscope for the presence or absence of crystalline inclusions. Protein gels were run of the samples to verify that the protein profiles between the cured and the uncured strains differed.

### EXAMPLE 5

### Examining Whether Bacillus thuringiensis Cry Gene Expression is Temporal and/or Affected by Polyadenylation

In this study, it was hypothesized that a given Bt strain with known insectidical activity was expected to have no difference in temporal expression of the insecticidal gene when analyzed using random reverse transcription primers and oligo(dT) reverse transcription primers. It was hypothesized that the expression levels of the Bacillus thuringiensis RNA correlate temporally with the cell stage/sporulation.

Bacillus thuringiensis Strain

A Bacillus thuringiensis strain, known as DP1019, has previously been shown to have insecticidal activity and to contain the gene cry9Db1. When expressed, the Cry9Db1 protein has been shown to have insecticidal activity similar to the DP1019 B. thuringiensis strain. The B. thuringiensis strain DP1019 was obtained from Pioneer Hi-Bred in Johnston, Iowa and it was cultured in terrific broth overnight at 30° C. with 250 rpm shaking The next day, the overnight culture was used to inoculate T3 expression medium, which was placed at 28° C. with shaking at 275 rpm. Starting 8 hours after the initial T3 culture was inoculated, and occurring every 4 hours until 32 hours post-inoculation, samples were observed microscopically to see where they were in their life cycle. At each timepoint, 1.0 ml samples of the Bt culture were harvested following Qiagen's RNAprotect Bacterial Reagent protocol #4 (catalog # 74524) and placed at −80 ° C.

### RNA Isolation and Analysis

RNA was isolated from the samples previously harvested following Qiagen's RNAprotect Bacterial Reagent protocol #4, followed by protocol #7 and using the optional on-column DNase treatment protocol found in Appendix B of the protocol. Total RNA concentrations were determined spectrophotometrically prior to the removal of DNA contamination by Ambion's TURBO DNA-free DNase treatment (catalog #AM1907). The total RNA samples were then run on an Agilent Bioanalyzer Nano6000 RNA chip to determine RNA quality and concentration, which were then normalized to 20 ng/μl.

### RT-PCR

The normalized RNA was used in reverse transcription reactions with Invitrogen's SuperScriptIII First-Strand Synthesis System for RT-PCR (catalog #18080-051), using both random hexamer primers and oligo(dT)₂₀ primers. 2.5 μl of first strand cDNA was used to set-up PCR reactions to detect a 966 by portion of the known insectidical cry9Db1 gene using Invitrogen's Platinum PCR SuperMix High Fidelity (catalog #12532-016) and gene specific PCR primers. The reactions were set-up following Invitrogen's protocol and cycled on a MJ Thermocycle. The RT-PCR products were run on an agarose+ethidium bromide gel to determine whether the RNA samples were positive for cry9Db1 (see Table 2).

TABLE 2
Cry9Db1 RT-PCR Reactions with Platinum Hi-Fi PCR (DP1019)

| Gel 1: Lane - Sample | Gel 2: Lane - Sample |
|---|---|
| 1 - ZipRuler 2-5 μl | 1 - ZipRuler 2-5 μl |
| 2 - 12 hr Random Hexamers | 2 - 12 hr Random No RT |
| 3 - 12 hr Oligo(dT)20 | 3 - 12 hr Oligo(dT) No RT |
| 4 - 16 hr #1 Random Hexamers | 4 - 16 hr #1 Random No RT |
| 5 - 16 hr #1 Oligo(dT)20 | 5 - 16 hr #1 Oligo(dT) No RT |
| 6 - 16 hr #2 Random Hexamers | 6 - 16 hr #2 Random No RT |
| 7 - 16 hr #2 Oligo(dT)20 | 7 - 16 hr #2 Oligo(dT) No RT |
| 8 - 20 hr #1 Random Hexamers | 8 - 20 hr #1 Random No RT |
| 9 - 20 hr #1 Oligo(dT)20 | 9 - 20 hr #1 Oligo(dT) No RT |
| 10 - 20 hr #2 Random Hexamers | 10 - 20 hr #2 Random No RT |
| 11 - 20 hr #2 Oligo(dT)20 | 11 - 20 hr #2 Oligo(dT) No RT |
| 12 - 24 hr #1 Random Hexamers | 12 - 24 hr #1 Random No RT |
| 13 - 24 hr #1 Oligo(dT)20 | 13 - 24 hr #1 Oligo(dT) No RT |
| 14 - 24 hr #2 Random Hexamers | 14 - 24 hr #2 Random No RT |
| 15 - 24 hr #2 Oligo(dT)20 | 15 - 24 hr #2 Oligo(dT) No RT |
| 16 - 28 hr Random Hexamers | 16 - 28 hr Random No RT |
| 17 - 28 hr Oligo(dT)20 | 17 - 28 hr Oligo(dT) No RT |
| 18 - 32 hr Random Hexamers | 18 - 32 hr Random No RT |
| 19 - 32 hr Oligo(dT)20 | 19 - 32 hr Oligo(dT) No RT |
| 20 - 20 hr B6 cured Random | 20 - 20 hr B6 cured Random No RT |
| Hexamers | |
| 21 - 20 hr B6 cured Oligo(dT)20 | 21 - 20 hr B6 cured Oligo(dT) No RT |
| 22 - 24 hr B6 cured Random | 22 - 24 hr B6 cured Random No RT |
| Hexamers | |
| 23 - 24 hr B6 cured Oligo(dT)20 | 23 - 24 hr B6 cured Oligo(dT) No RT |
| 24 - ZipRuler 2-5 μl | 24 - ZipRuler 2-5 μl |
| 25 - 10 μl DP1019-B6 cured DNA | 25 - NTC Random hexamers |
| control | |
| 26 - 10 μl DP1019 uncured DNA + | 26 - NTC Oligo(dT)20 |
| control | |

### Statistical Analysis

Binomial probability distribution analysis was used to determine if there was a significant different between the different primer groups. The degree of freedom was set at 1 and alpha was 0.05.

Results

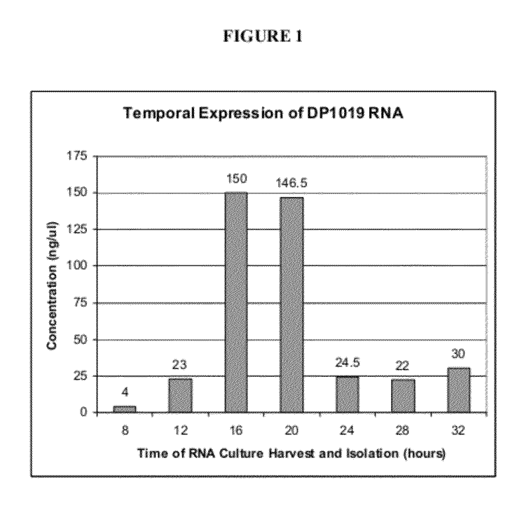

The B. thuringiensis RNA concentration varied widely with time (FIG. 1), as did the development stage of the cells. At 8 hours post-inoculation, the cells were still in vegetative growth and did not produce much RNA, so the sample was excluded from further analysis. At 12 hours post-inoculation, the culture was in the very early stages of sporulation. The 16 hour and 20 hour samples were composed of cells containing crystalline inclusions and spores, with more being contained in the latter. The density of intact cells started to decrease with the 24 hour sample and continued to decrease with the latter time-points. There was reduced cell density with free spores and crystals present alongside spore and crystal containing intact cells in the 24 hour sample. Even fewer intact cells were observed on the microscope slide of the 32 hour sample, as compared to the 28 hour sample, but both had an increased abundance of free spores and crystals present in culture.

The results of the RT-PCR reactions for the DP1019 RNA timepoints are shown in Table 3. “No RT” reactions were also set-up using the same RNA samples and all were negative, with the exception of DP1019 sample 24 hour #1. The no reverse transcriptase reactions for this sample produced a faint positive band with both the random hexamer primer and the oligo(dT)₂₀ primer. The binomial probability distribution analysis for the two groups yielded the same result. The p value was equal to 1.0, which is significantly less than the critical value of 3.8. There is no significant difference between the random hexamer and the oligo(dT)₂₀ sets of data.

TABLE 3
RT-PCR Results for the Amplification of 966 bp Portion of cry9Db1 using DP1019
Bacillus thuringiensis RNA

| | Time-point Results | | | | | | | | | Totals | |
|---|---|---|---|---|---|---|---|---|---|---|---|
| RT Primer | 12 h | 16 h #1 | 16 h #2 | 20 h #1 | 20 h #2 | 24 h #1 | 24 h #2 | 28 h | 32 h | Pos. | Neg. |
| Random hexamers | + | + | + | + | + | + | + | + | + | 9 | 0 |
| Oligo(dT)₂₀ | + | + | + | + | + | + | + | + | + | 9 | 0 |

The peak in RNA expression occurs at 16 hours and 20 hours after inoculation of T3 media and is probably related to the various sigma factors involved in RNA transcription controlling expression during sporulation. With approximately 6× higher expression at 16 and 20 hours than at 12, 24, 28, and 32-hours (FIG. 1), the RNA of strain DP1019 appears to be under the control of sporulation specific factors (Cheng et al. (1999) Appl. Environ. Microbiol. 65:1849-1853). B. thuringiensis sigma factors 35 and 28 are homologous to B. subtilis sigma factors E and K, which showed peaks in expression 4 hours apart (Zhang et al. (1998) Nucleic Acids Res. 26:1288-1293). The cry9Db1 gene expression probably correlates with the overall RNA expression levels, indicating that the gene's expression is likely under the control of both sigma 35 and sigma 28. The sigma factor promoters likely overlap, as with BtI and BtII and cry1A expression (Sedlak et al. (2000) J. Bacteriol. 182:734-741). The onset of sigma 35 expression at early to mid-stages of sporulation, followed by mid-to late-sporulation expression of sigma 28, is in line with the RNA abundance data and observations of the culture (Brown and Whiteley, (1990) J. Bacteriol. 172:6682-6688). Different expression patterns have been reported for other cry genes and range from vegetative expression, to high expression in the log phase, to the beginning of stationary phase, and through mid-sporulation, however cry9Db1 expression is present throughout the sporulation process and peaks around mid-sporulation, which is indicative of sigma 35 and sigma 28 sequential expression (Yoshisue et al. (1993) J. Bacteriol. 175:2750-2753; Agaisse and Lereclus (1994) J. Bacteriol. 176:4734-4741; Guidelli-Thuler et al. (2009) Sci. Agric. 66:403-409).

All experimental samples analyzed produced a positive RT-PCR signal (Table 3), indicating that the cry9Db1 transcript is polyadenylated, but to what degree cannot be determined by the data. Fewer rounds of PCR on the cDNA may have provided a better look at the amounts of polyadenylated transcript, as the RT-PCR products were all very intense bands on the agarose gel (data not shown). A study done on Mycobacterium tuberculosis indicated that not all mRNAs were equally polyadenylated and that as few as 2% of transcripts may have poly-A tails (Lakey et al. (2002) Microbiology 148:2567-2572).

Based on the collected data, it can be concluded that Bacillus thuringiensis polyadenylated RNA can be used in insecticidal gene discovery. Traditional approaches for the detection of new insecticidal genes and proteins include protein purification, PCR-RFLP, hybridizations and immunoblotting (Guerchicoff et al. (1997) Appl. Environ. Microbiol. 63:2716-2721; Donovan et al. (2006) Appl Microbiol Biotechnol. 72:713-719; Liu et al. (2009) Front. Agric. China 2009, 3, 159-163). When examining a strain with bioactivity, analysis of the transcripts may prove to be an easier path forward, as compared to other methodologies to gene and protein identification. Isolating B. thuringiensis RNA and using the poly-A tails for oligo(dT) priming or mRNA isolation, followed by transcriptome sequencing, may provide a more efficient method for novel insecticidal gene discovery versus traditional methods.

### EXAMPLE 6

### cry9Ed1 Oligo(dT)₂₀ Assay

Assays described in Example 5 for cry9Db1 were repeated for a second cry gene, cry9Ed1. For these experiments, RNA samples from 16- and 20-hour post-inoculation samples were pooled and then divided for subsequent reactions. Invitrogen's SuperScript III kit was used for RT-PCR. The expected RT-PCR product of 1203 basepairs was observed in samples obtained at 16- and 20-hours post-inoculation with random hexamer and oligo(dT)₂₀ primers while no bands were observed on a 1% agarose gel (stained with ethidium bromide) in no-RT control lanes (data not shown). These data demonstrated that, as described in Example 5 for cry9Db1, the 16- and 20-hour transcripts for cry9Ed1 were polyadenylated.

That which is claimed:

1. A method for identifying at least one polynucleotide encoding a polypeptide having pesticidal activity, said method comprising the steps of:
(a) providing a bacterial strain having or suspected of having a pesticide-encoding nucleotide sequence;
(b) curing a plasmid from the bacterial strain creating a control strain;
(c) using substrative hybridization of the polynucleotide sequences of the control strain and the bacterial strain having the pesticide-encoding nucleotide sequence (the target strain) to identify sequences suspected of encoding the polypeptide having pesticidal activity; and,
(d) identifying at least one polypeptide having pesticidal activity.

2. The method of claim 1 wherein said substrative hybridization is in vitro substrative hybridization.

3. The method of claim 1, wherein said substrative hybridization is in silico substrative hybridization.

4. The method of claim 2, wherein said method comprises:
(a) purifying mRNA molecules from the control strain;
(b) purifying mRNA molecules from the target strain;
(c) performing subtractive hybridization with the mRNA molecules of the control strain and the mRNA molecules of the target strain to isolate unique mRNA molecules of the bacterial strain of the target strain;
(d) generating a cDNA expression library from the unique mRNA molecules;
(e) screening the cDNA expression library for at least one cDNA encoding a polypeptide having pesticidal activity.

5. The method of claim 3, wherein said method comprises:
(a) sequencing the genome of the control strain;
(b) sequencing the genome of the target strain;
(c) identifying unique sequences; and,
(d) analyzing unique sequences for those that encode a pesticidal polypeptide.

6. A method for identifying at least one polynucleotide encoding a polypeptide having pesticidal activity, said method comprising the steps of:
(a) providing a bacterial strain having or suspected of having a pesticide-encoding nucleotide sequence;
(b) curing the plasmid from the bacterial strain;
(c) purifying mRNA molecules from the bacterial strain of (a);
(d) purifying mRNA molecules from the bacterial strain of (b);
(e) performing subtractive hybridization with the mRNA molecules of (c) and the mRNA molecules of (d) to isolate unique mRNA molecules of the bacterial strain of (a);
(f) generating a cDNA expression library from the unique mRNA molecules of (e);
(g) screening the cDNA expression library for at least one cDNA encoding a polypeptide having pesticidal activity.

7. The method of claim 6, further comprising sequencing the cDNA encoding the polypeptide having pesticidal activity.

8. The method of claim 6, wherein the bacterial strain is a Bacillus spp.

9. The method of claim 8, wherein the Bacillus spp. is a Bacillus thuringiensis strain.

10. The method of claim 6, wherein step (b) comprises heating cells of the bacterial strain to a temperature between about 35° C. and about 45° C.

11. The method of claim 10, wherein the temperature is about 40° C.

12. The method of claim 6, wherein step (b) further comprises verifying that the bacterial strain lacks insecticidal activity.

13. The method of claim 12, wherein the verifying step comprises performing an in vitro insect bioassay.

14. The method of claim 6, wherein steps (c) and (d) comprise growing the bacterial strains of (a) and (b) under conditions that induce expression of the pesticide-encoding plasmid.

15. The method of claim 6, wherein step (f) comprises amplifying mRNA molecules using oligo(dT) primers.

16. The method of claim 6, wherein step (f) comprises:
(a″) reverse transcribing the unique nucleic acid molecules of (e) to produce a DNA/RNA hybrid;
(b″) removing the RNA from the DNA/RNA hybrid;
(c″) performing second strand synthesis to produce unique cDNA; and
(d″) inserting the unique cDNA into an expression plasmid.

17. The method of claim 6, wherein step (g) comprises performing an in vitro insect bioassay.
